(19) United States
(12) Patent Application Publication
(10) Pub. No.: US 2009/0170906 A1
(43) Pub. Date: Jul. 2, 2009

(54) HCV INHIBITORS

(76) Inventors: Kristjan Gudmundsson, Durham, NC (US); Vicente Samano, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/719,899

(22) PCT Filed: Nov. 14, 2005
(86) PCT No.: PCT/US05/41089

§ 371 Date: May 22, 2007

Related U.S. Application Data
(60) Provisional application No. 60/629,905, filed on Nov. 22, 2004.

(51) Int. Cl. A61K 31/4439; A61K 31/403

(52) U.S. Cl. 514/339; 514/411

(57) ABSTRACT

The present invention relates to compounds that are useful in the treatment of viruses belonging to Flaviviridae, including flaviviruses, pestiviruses, and hepaciviruses. The invention includes compounds useful for the treatment or prophylaxis of dengue fever, yellow fever, West Nile virus, and HCV.

### FIELD OF THE INVENTION

The present invention relates to compounds useful as anti-viral agents. The present invention relates to compounds that are useful in the treatment of viruses belonging to Flaviviridae, including flaviviruses, pestiviruses, and hepaciviruses. The invention includes compounds useful for the treatment or prophylaxis of dengue fever, yellow fever, West Nile virus, and HCV.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA—A Publication of the RNA Society. 1(5): 526-537, 1995 July). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N.Y.). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

Based on the foregoing, there exists a significant need to identify synthetic or biological compounds for their ability to inhibit HCV.

### SUMMARY OF THE INVENTION

The present invention includes a method for the treatment or prophylaxis of Flaviviridae viruses through administration of a compound of formula (I):

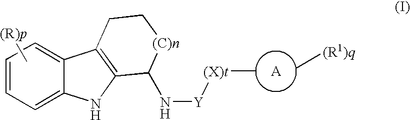

wherein: n is 0, 1, or 2; t is 0 or 1; X is —NH—, —O—, —R¹⁰—, —OR¹⁰—, —R¹⁰O—, —R¹⁰OR¹⁰—, —NR¹⁰—, —R¹⁰N—, —R¹⁰NR¹⁰—, —R¹⁰S(O)ₘ—, or —R¹⁰S(O)ₘR¹⁰—; Y is —C(O)— or —S(O)ₘ—; each R is the same or different and is independently selected from the group consisting of halogen, haloalkyl, alkyl, alkenyl, alkynyl, cycloalkyl, cycloalkenyl, —R¹⁰ cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each R¹ is the same or different and is independently selected from the group consisting of halogen, haloalkyl, alkyl, alkenyl, alkynyl, cycloalkyl, cycloalkenyl, —R¹⁰ cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
p and q are each independently selected from 0, 1, 2, 3, 4, or 5;
each of R² and R³ are the same or different and are independently selected from the group consisting of H, alkyl, alkenyl, alkynyl, cycloalkyl, cycloalkenyl, —R¹⁰cycloalkyl, —R¹⁰H, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group; Het represents a 5- or 6-membered heterocyclyl or heteroaryl group; ring A is aryl or heteroaryl; provided that when the A ring is aryl, t is 0, and Y is SO₂, then p is not 0;
including salts, solvates and physiologically functional derivatives thereof.

Preferably, the Flaviviridae virus is a flavivirus, a pestivirus, or a hepacivirus. More particularly, the virus is associated with a human disease selected from dengue fever, yellow fever, west nile virus, and HCV. Further, preferably the method is for the treatment or prophylaxis of HCV infection.

Preferably alkyl is C₁-C₆ alkyl, alkoxy is C₁-C₆ alkoxy, haloalkyl is C₁-C₆ haloalkyl, alkylene is C₁-C₆ alkylene, and alkenylene is C₁-C₆ alkenylene.

In one embodiment t is 0 and Y is —C(O)—. In another embodiment t is 0 and Y is —S(O)ₘ—. In another embodiment t is 1, Y is —C(O)—, and X is —NH—, —O—, —R¹⁰—, or —OR¹⁰—. In another embodiment t is 1, Y is —S(O)ₘ—, and X is —NH—, —O—, —R¹⁰—, or —OR¹⁰—.

In one embodiment n is 1.

In one embodiment p is 1 or more and R is selected from halogen, alkyl, haloalkyl, —OR², —NR²R³, —C(O)R², —CO₂R², cyano, nitro, or azido. Preferably R is halogen, alkyl, haloalkyl. Preferably R is substituted para to the depicted N atom. Preferably R is halogen. Preferably R is Br or Cl.

In one embodiment q is 1 or more and R¹ is selected from halogen, alkyl, haloalkyl, —OR², —NR²R³, —C(O)R², —CO₂R², Ay, Het, cyano, nitro, or azido. Preferably R¹ is selected from halogen, alkyl, haloalkyl, —OR², —NR²R³, —C(O)R², —CO₂R², or cyano. Preferably R² and R³ each are C₁-C₆ alkyl. Preferably R¹ is selected from halogen, alkyl, or —OR². Preferably halogen is fluoro or chloro, alkyl is methyl, and —OR² is alkoxy.

In one embodiment the A ring is aryl. Preferably the A ring is phenyl. Preferably q is 1 or more and R¹ is selected from halogen, alkyl, haloalkyl, —OR², —NR²R³, —C(O)R², —CO₂R², Ay, Het, cyano, nitro, or azido. More preferably q is 1 or more and R¹ is selected from halogen, alkyl, haloalkyl, —OR², —NR²R³, —C(O)R², —CO₂R², or cyano.

In one embodiment the A ring is heteroaryl. Preferably the heteroaryl is pyridyl. Preferably q is 0 or 1. Further, when q is 1 then R¹ is selected from halogen, alkyl, haloalkyl, —OR², —NR²R³, —C(O)R², —CO₂R², Ay, Het, cyano, nitro, or azido. More preferably when q is 1 then R¹ is selected from halogen, alkyl, haloalkyl, —OR², —NR²R³, —C(O)R², —CO₂R², or cyano.

In one embodiment p is 1, R is halogen, n is 1, Y is —C(O)—, t is 0, ring A is heteroaryl, and q is 0. Preferably R is chloro and ring A is pyridyl.

In one embodiment method includes the use of a compound selected from:

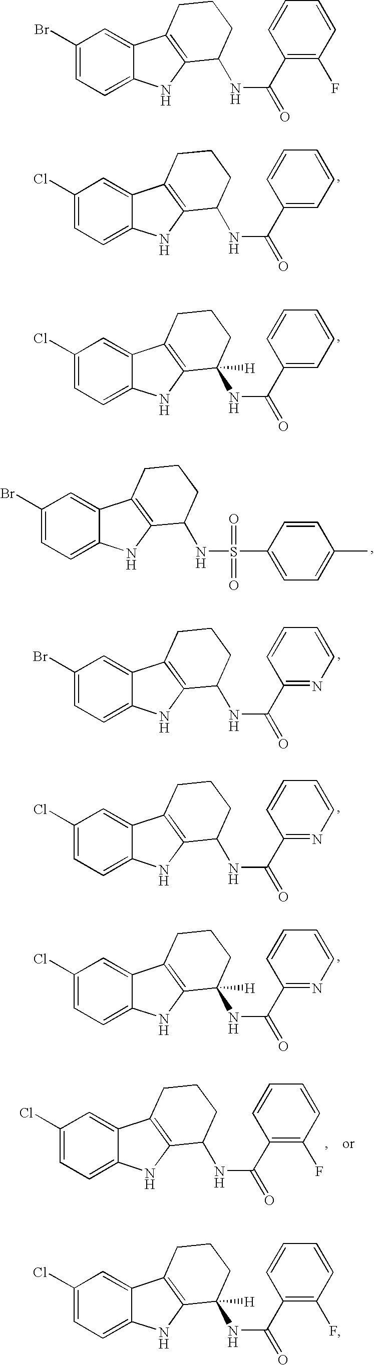

including salts, solvates, and physiologically functional derivatives thereof.

The present invention includes the use of a compound of formula (I):

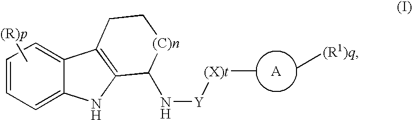

wherein the variables are as defined in the manufacture of a medicament for use in the treatment or prophylaxis of viruses belonging to Flaviviridae.

Preferably the virus is a flavivirus, a pestivirus, or a hepacivirus. Further preferably the disease or condition is dengue fever, yellow fever, west nile virus, or HCV. Still further preferably the condition or disorder is HCV.

The present invention also includes novel compounds. One aspect of the present invention includes a compound selected from:

One aspect of the present invention includes one or more of:
N-(6-Chloro-2,3,4,9-tetrahydro-1H-carbazol-1-yl)-2-pyrazinecarboxamide;
N-(6-Chloro-2,3,4,9-tetrahydro-1H-carbazol-1-yl)-6-methyl-2-pyridinecarboxamide;
N-(6-Chloro-2,3,4,9-tetrahydro-1H-carbazol-1-yl)-5-isoxazolecarboxamide;
N-(6-Chloro-2,3,4,9-tetrahydro-1H-carbazol-1-yl)-2-furancarboxamide;
N-(6-Chloro-2,3,4,9-tetrahydro-1H-carbazol-1-yl)-3,5-dimethyl-4-isoxazolecarboxamide;
N-(6-Chloro-2,3,4,9-tetrahydro-1H-carbazol-1-yl)-6-fluoro-2-pyridinecarboxamide;
N-(6-Chloro-2,3,4,9-tetrahydro-1H-carbazol-1-yl)-3-methylpyridine-2-carboxamide;
N-[2-({2-[(2-Aminoethyl)oxy]ethyl}oxy)ethyl]-N′-(6-chloro-2,3,4,9-tetrahydro-1H-carbazol-1-yl)-1,4-benzenedicarboxamide;
Methyl 6-{[(6-chloro-2,3,4,9-tetrahydro-1H-carbazol-1-yl)amino]carbonyl}-3-pyridinecarboxylate;
6-{[(6-Chloro-2,3,4,9-tetrahydro-1H-carbazol-1-yl)amino]carbonyl}-3-pyridinecarboxylic acid;
1,1-Dimethylethyl [2-({2-[(2-{[(6-{[(6-chloro-2,3,4,9-tetrahydro-1H-carbazol-1-yl)amino]carbonyl}-3-pyridinyl)carbonyl]amino}ethyl)oxy]ethyl}oxy)ethyl]carbamate;
N⁵-[2-({2-[(2-Aminoethyl)oxy]ethyl}oxy)ethyl]-N²-(6-chloro-2,3,4,9-tetrahydro-1H-carbazol-1-yl)-2,5-pyridinedicarboxamide; and
N²-(6-Chloro-2,3,4,9-tetrahydro-1H-carbazol-1-yl)-N⁵-(2-{[2-({2-[(phenylcarbonyl)amino]ethyl}oxy)ethyl]oxy}ethyl)-2,5-pyridinedicarboxamide,
   including salts, solvates, and physiologically functional derivatives thereof.

Another aspect of the present invention includes the above-referenced compounds substantially as defined with reference to any one of the Examples.

Another aspect of the present invention includes pharmaceutical compositions comprising a compound as described herein, and a pharmaceutically acceptable carrier. Preferably the compound is for use as an active therapeutic substance.

Another aspect of the present invention includes a compound as herein described for use in the treatment or prophylaxis of diseases and conditions caused by viruses belonging to Flaviviridae. Preferably the virus is a flavivirus, a pestivirus, or a hepacivirus. Preferably the disease or condition is dengue fever, yellow fever, west nile virus, or HCV. More specifically the condition or disease is HCV.

Another aspect of the present invention includes the use of a compound as herein described in the manufacture of a medicament for use in the treatment or prophylaxis of viruses belonging to Flaviviridae. Preferably the virus is a flavivirus, a pestivirus, or a hepacivirus. Preferably the disease or condition is dengue fever, yellow fever, west nile virus, or HCV. More specifically the disease or condition is HCV.

Another aspect of the present invention includes a method for the treatment or prophylaxis of viruses belonging to Flaviviridae that includes administration of a compound as herein described. Preferably the virus is a flaviviruses, a pestiviruses, or a hepaciviruses. Preferably the virus is associated with a human disease selected from dengue fever, yellow fever, west nile virus, and HCV. More specifically the method is for the treatment or prophylaxis of HCV infection.

As noted herein, p and q are each independently defined as 0, 1, 2, 3, 4, or 5. Notably, as will be appreciated by those skilled in the art, the value(s) of p and/or q should not exceed the substitutable positions on the depicted rings.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, n-pentyl, and the like.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to a straight or branched chain divalent hydrocarbon radical, preferably having from one to ten carbon atoms. Alkylene groups as defined herein may optionally be substituted. Examples of “alkylene” as used herein include, but are not limited to, methylene, ethylene, n-propylene, n-butylene.

As used herein, the term “alkenylene” refers to a straight or branched chain divalent hydrocarbon radical, preferably having from one to ten carbon atoms, containing one or more carbon-to-carbon double bonds that may be optionally substituted. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds that may be optionally substituted. Examples include, but are not limited to, ethynylene and the like.

As used herein, the term “cycloalkyl” refers to an optionally substituted non-aromatic cyclic hydrocarbon ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and substituted versions thereof. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, cycloheptenyl, and substituted versions thereof.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, cycloheptylene, and the like.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, cycloheptenylene, and the like.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. Preferably the ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, 1-naphthyl, and the like.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, pyrazolopyrimidinyl, and the like.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to the group —OR^{a}, where R^{a} is alkyl as defined above.

As used herein the term “alkoxycarbonyl” refers to groups such as:

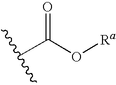

where the R^{a} represents an alkyl group as herein defined.

As used herein the term “aryloxycarbonyl” refers to groups such as:

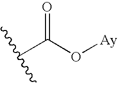

where the Ay represents an aryl group as herein defined.

As used herein the term “heteroaryloxycarbonyl” refers to groups such as:

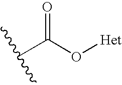

where the Het represents a heteroaryl group as herein defined.

As used herein the term “nitro” refers to the group —NO₂.

As used herein the term “cyano” refers to the group —CN.

As used herein the term “azido” refers to the group —N₃.

As used herein the term “acyl” refers to the group R^{b}C(O)—, where R^{b} is alkyl, aryl, heteroaryl, or heterocyclyl, as each is defined herein.

As used herein the term “oxo” refers to the group ═O.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

Exemplary optional substituent groups include acyl; alkyl; alkenyl; alkynyl; alkylsulfonyl; alkoxy; alkoxycarbonyl; cyano; halogen; haloalkyl; hydroxy; nitro; aryl, which may be further substituted with acyl, alkoxy, alkyl, alkenyl, alkynyl, alkylsulfonyl, cyano, halogen, haloalkyl, hydroxy, or nitro; heteroaryl, which may be further substituted with acyl, alkoxy, alkyl, alkenyl, alkynyl, alkylsulfonyl, cyano, halogen, haloalkyl, hydroxy, or nitro; arylsulfonyl, which may be further substituted with acyl, alkoxy, alkyl, alkenyl, alkynyl, alkylsulfonyl, cyano, halogen, haloalkyl, hydroxy, or nitro; heteroarylsulfonyl, which may be further substituted with acyl, alkoxy, alkyl, alkenyl, alkynyl, alkylsulfonyl, cyano, halogen, haloalkyl, hydroxy, or nitro; aryloxy, which may be further substituted with acyl, alkoxy, alkyl, alkenyl, alkynyl, alkylsulfonyl, cyano, halogen, haloalkyl, hydroxy, or nitro; heteroaryloxy, which may be further substituted with acyl, alkoxy, alkyl, alkenyl, alkynyl, alkylsulfonyl, cyano, halogen, haloalkyl, hydroxy, or nitro; aryloxycarbonyl, which may be further substituted with acyl, alkoxy, alkyl, alkenyl, alkynyl, alkylsulfonyl, cyano, halogen, haloalkyl, hydroxy, or nitro; heteroaryloxycarbonyl, which may be further substituted with acyl, alkoxy, alkyl, alkenyl, alkynyl, alkylsulfonyl, cyano, halogen, haloalkyl, hydroxy, or nitro; or —N(R*)₂; where for each occurrence R* is independently selected from H, alkyl, alkenyl, alkynyl, cycloalkyl, heterocyclyl, aryl, aralkyl, heteroaryl, heteroaralkyl, alkylsulfonyl, arylsulfonyl, or heteroarylsulfonyl, where each occurrence of such aryl or heteroaryl may be substituted with one or more acyl, alkoxy, alkyl, alkenyl, alkylsulfonyl, cyano, halogen, haloalkyl, hydroxy, or nitro, or the two R*s may combine to form a ring, optionally having additional heteroatoms, optionally having one or more degrees of unsaturation, and optionally being further substituted with acyl, alkoxy, alkyl, alkenyl, alkynyl, alkylsulfonyl, cyano, halogen, haloalkyl, hydroxy, or nitro.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. Treatment will depend upon the nature and type of viral infection. The present invention may be combined with other medical therapies including a variety of cytotoxic or antiviral agents. For example, and not meant to limit the invention, the compounds of the present invention may be combined with other therapeutic agents for example immune therapies (such as interferon), therapeutic vaccines, antifibrotic agents, anti-inflammatory agents (such as corticosteroids or NSAIDs), bronchodilators such as beta-2 adrenergic agonists and xanthines (such as theophylline), mucolytic agents, anti-muscarinics, anti-leukotrienes, inhibitors of cell adhesion (such as ICAM antagonists), anti-oxidants (such as N-acetylcysteine), cytokine agonists, cytokine antagonists, lung surfactants and/or antimicrobial and anti-viral agents (such as ribavirin and amantidine). The compositions according to the invention may also be used in combination with gene replacement therapy.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
RT (room temperature); h (hours);
min (minutes); TLC (thin layer chromatography);
mp (melting point); RP (reverse phase);
Tr (retention time); TFA (trifluoroacetic acid);
TEA (triethylamine); THF (tetrahydrofuran);
TFAA (trifluoroacetic anhydride); CD3OD (deuterated methanol);
CDCl₃ (deuterated chloroform); DMSO (dimethylsulfoxide);
SiO₂ (silica); atm (atmosphere);
EtOAc (ethyl acetate); CHCl3 (chloroform);
HCl (hydrochloric acid); Ac (acetyl);
DMF (N,N-dimethylformamide); Me (methyl);
Cs2CO3 (cesium carbonate); EtOH (ethanol);
Et (ethyl); tBu (tert-butyl);
MeOH (methanol).

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds were assigned by Ab Inito Vibrational Circular Dichroism (VCD) Spectroscopy. The experimental VCD spectrum were acquired in CDCl₃ using a Bomem Chirall® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration.

Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A.11.4, M. J. Frisch et al., Gaussian, Inc., Pittsburgh Pa., 2002.

Compounds of formula (I) wherein variables are as defined above and LV is a leaving group, namely halogen (F, Cl, Br, I), may be conveniently prepared by the process outlined in Scheme 1 below:

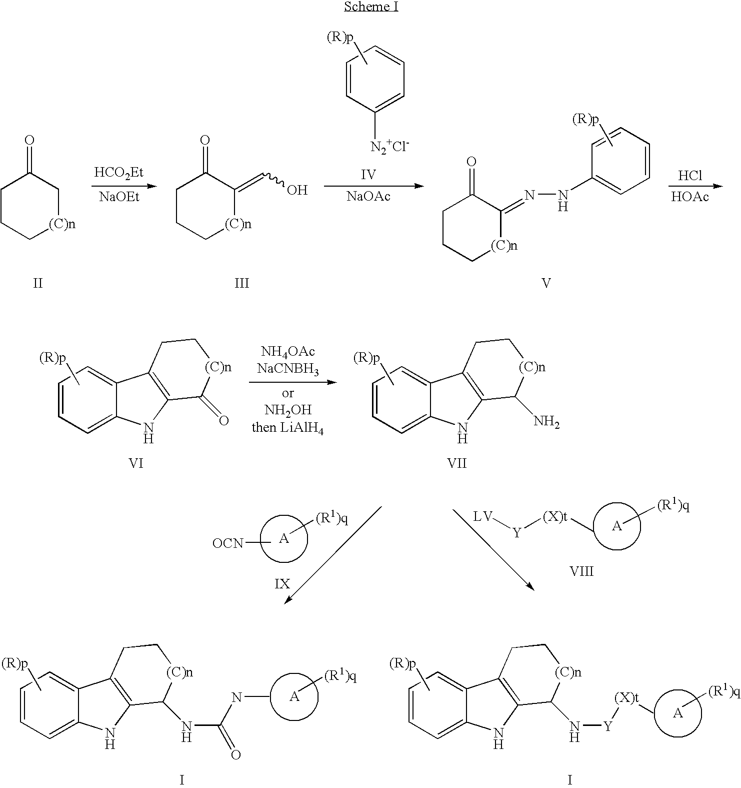

Generally, the process for preparing the compounds of formula (I), where LV is a leaving group as defined above (all formulas and variables as defined above) comprises the steps of:
a) reacting a compound of formula (II) with ethyl formate;
b) reacting the compound of formula (III) with diazacompound of formula (IV);
c) indolizing the compound of formula (V) to prepare a compound of formula (VI);
d) reductive amination of compound of formula (VI) to form compound of formula (VII); and
e) forming compounds of formula (I) from compound (VII) by reaction with compound of formula (VIII); or alternatively
f) forming compounds of formula (I) where Y is CO and X is NH via reaction of compound of formula (VII) with compound of formula (IX).

More specifically, a compound of formula (I) wherein all variables are as defined above can be prepared reacting the compound of formula (VII) with a compound of formula (VIII):

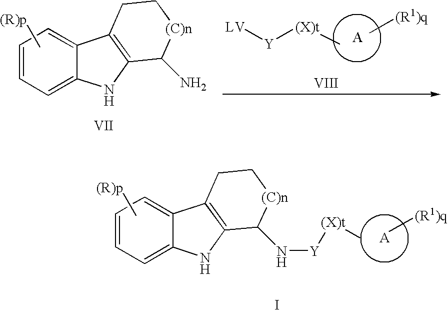

The reaction may be carried out by adding compound of formula (VIII) to a compound of formula (VII) in a suitable solvent, optionally in the presence of base, and optionally with heating. Suitable solvents include tetrahydrofuran, dichloromethane, N,N-dimethylformamide, pyridine, dioxane, diethyl ether, acetonitrile, toluene, and the like. Suitable bases include triethylamine, diisopropylethylamine, pyridine, dimethylaminopyridine, and the like. As will be appreciated by those skilled in the art, compounds of formula (VIII) are commercially available or can be prepared according to literature methods.

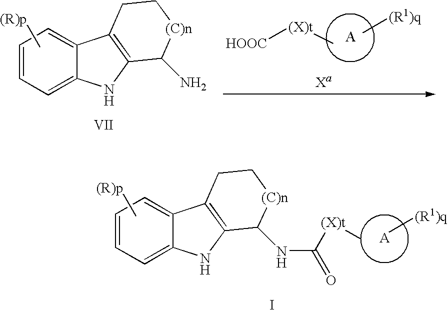

Additionally, as will be appreciated by those skilled in the art, a compound of formula (I) where Y is —C(O)— can also be formed by coupling an amine of formula (VII) and an acid of formula (X^{a}). Any set of standard coupling conditions as are known to those skilled in the art may be used for this coupling.

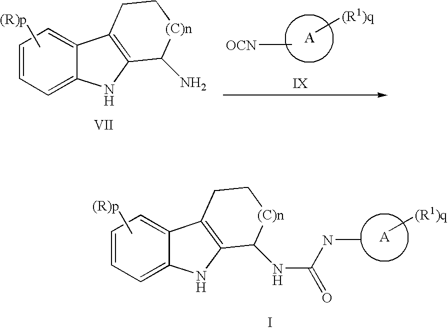

Alternatively, a compound of formula (I) where Y is —CO— and X is —NH— can be formed by the treatment of a compound of formula (VII) with an isocyanate compound of formula (IX) in a suitable solvent, optionally with heating. Suitable solvents include tetrahydrofuran and the like. Isocyanates of formula (IX) are commercially available or may be prepared by literature methods that are appreciated by those skilled in the art.

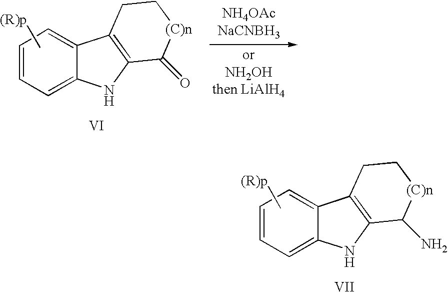

An amine compound of formula (VII) can be formed from a compound of formula (VI). Treatment of a compound of formula (VI) in an inert solvent with ammonium salt and a reductive agent, optionally with heating, gives an amine of formula (VII). Suitable solvents include but are not limited to, methanol, ethanol, dichloromethane, dichloroethane, and the like. Suitable reductive agents include but are not limited to sodium cyanoborohydride, sodium triacetoxyborohydride, sodium borohydride, and the like. Suitable ammonium salts include but are not limited to ammonium acetate, ammonium formate and the like.

An amine of formula (VII) can also be formed by treatment of a compound of formula (VI) with hydroxylamine, followed by reduction with suitable reductive agents which include, but are not limited to, lithium aluminium hydride and the like.

Compounds of formula (VI) are prepared in a similar fashion as described in the literature (J. Med. Chem. 1973, 16, 425 and J. Org. Chem. 1968, 32, 1265), herein incorporated by reference to the extent of such teaching.

A compound of formula (I) can be converted to another compound of formula (I) by methods appreciated by those skilled in the art.

### EXAMPLES

### Example 1

### 6-Chloro-2,3,4,9-tetrahydro-1H-carbazol-1-one

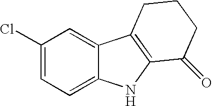

### a) Cyclohexane-1,2-dione (4-chlorophenyl)hydrazone

To a cold (0° C.) solution of 4-chloroaniline (5.6 g, 44 mmol) in concentrated hydrochloric acid (5 mL) was added sodium nitrite (3.0 g, 44 mmol) dissolved in water (10 mL) portionwise over 20 minutes. The mixture was stirred at 0° C. for 30 minutes. In a separate flask, a cool solution of 2-(hydroxymethylene)cyclohexanone (Organic Syntheses, Collective Vol 4, 1963, pg. 536) (5.0 g, 40 mmol) in methanol (30 mL) was treated with a solution of sodium acetate (8.3 g, 101 mmol) in water (25 mL). The mixture was stirred at 0° C. for 20 minutes and the diazonium salt slurry was added. The combined mixture was stirred for 10-15 minutes, collected by filtration, triturated with ethanol, and collected by filtration to give cyclohexane-1,2-dione (4-chlorophenyl)hydrazone (4.6 g, 49% yield) as a yellow solid. ¹H-NMR (DMSO-d₆): δ 9.93 (s, 1H), 7.29 (m, 4H), 2.55 (m, 2H), 2.40 (m, 2H), 1.84-1.75 (m, 4H).

### b) 6-Chloro-2,3,4,9-tetrahydro-1H-carbazol-1-one

A solution of cyclohexane-1,2-dione (4-chlorophenyl)hydrazone (2.3 g, 9.7 mmol) in hydrochloric acid (2 mL) and acetic acid (8 mL) was heated at 120° C. for 20 minutes. The mixture was cooled slightly and treated with ice water. The resulting precipitate was collected by filtration to give 6-chloro-2,3,4,9-tetrahydro-1H-carbazol-1-one (1.9 g, 88% yield) as brown solid. ¹H-NMR (DMSO-d₆): δ 11.78 (s, 1H), 7.75 (m, 1H), 7.38 (d, 1H), 7.28 (dd, 1H), 2.92 (t, 2H), 2.55 (t, 2H), 2.13 (q, 2H); MS m/z 220 (M+1).

### Example 2

### 6-Chloro-2,3,4,9-tetrahydro-1H-carbazol-1-amine

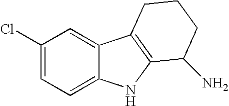

To a solution of 6-chloro-2,3,4,9-tetrahydro-1H-carbazol-1-one (500 mg, 2.3 mmol) and ammonium acetate (1.8 g, 23 mmol) in methanol (9 mL) was added sodium cyanoborohydride (720 mg, 11.5 mmol). After heating at 60° C. for 15 hours, the mixture was cooled and treated with concentrated hydrochloric acid until pH=1. The organics were removed under reduced pressure and the resulting precipitate was collected by filtration, dissolved in ethyl acetate and methanol, and washed with saturated aqueous sodium carbonate. The phases were separated and the organic phase was concentrated to yield 6-chloro-2,3,4,9-tetrahydro-1H-carbazol-1-amine (260 mg, 52% yield) as a light brown solid. ¹H-NMR (DMSO-d₆): δ 10.90 (s, 1H), 7.34 (m, 1H), 7.27 (d, 1H), 6.97 (dd, 1H), 3.90 (t, 1H), 2.54 (m, 2H), 2.04-1.89 (m, 2H), 1.66 (m, 1H), 1.50 (m, 1H); MS m/z 221 (M+1).

### Example 3

### 6-Bromo-2,3,4,9-tetrahydro-1H-carbazol-1-one

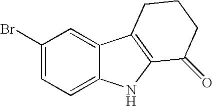

6-Bromo-2,3,4,9-tetrahydro-1H-carbazol-1-one was prepared from bromoaniline and 2-(hydroxymethylene)cyclohexanone in a similar manner as described in Example 1 to give a brown solid. ¹H-NMR (CDCl₃): δ 8.79 (s, 1H), 7.80 (s, 1H), 7.44 (d, 1H), 7.30, (d, 1H), 2.97 (t, 2H), 2.66 (t, 2H), 2.27 (quint, 2H); MS m/z 265 (M+1).

### Example 4

### 6-Bromo-2,3,4,9-tetrahydro-1H-carbazol-1-amine

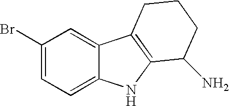

6-Bromo-2,3,4,9-tetrahydro-1H-carbazol-1-amine was prepared in a similar manner as described in Example 2 to give a solid. ¹H-NMR (CDCl₃): δ 8.58 (s, 1H), 7.55 (s, 1H), 7.20 (m, 2H), 4.12 (t, 1H), 2.70 (t, 2H), 2.24 (m, 1H), 2.05 (m, 1H), 1.92 (m, 3H), 1.66 (m, 1H); MS m/z 266 (M+1).

### Example 5

### 6-Methyl-2,3,4,9-tetrahydro-1H-carbazol-1-one

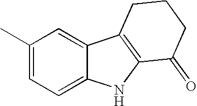

6-Methyl-2,3,4,9-tetrahydro-1H-carbazol-1-one was prepared from p-toluidine and 2-(hydroxymethylene)cyclohexanone in a similar manner as described in Example 1 to give a tan solid. ¹H-NMR (CDCl₃): δ 8.65 (s, 1H), 7.43 (s, 1H), 7.30 (d, 1H), 7.20 (d, 1H), 2.98 (t, 2H), 2.65 (t, 2H), 2.45 (s, 3H), 2.26 (quint, 2H); MS m/z 220 (M+1).

### Example 6

### 6-Methyl-2,3,4,9-tetrahydro-1H-carbazol-1-amine

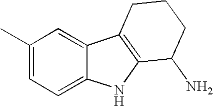

6-Methyl-2,3,4,9-tetrahydro-1H-carbazol-1-amine was prepared in a similar manner as described herein to give a solid. ¹H-NMR (DMSO-d₆): δ 10.5 (s, 1H), 7.15 (d, 1H), 7.11 (s, 1H), 6.81 (d, 1H), 3.98 (t, 1H), 3.30 (s, 2H), 2.53 (t, 2H), 2.32 (s, 3H), 2.02 (m, 1H), 1.90 (m, 1H), 1.68 (m, 1H), 1.65 (m, 1H); MS m/z 201 (M+1).

### Example 7

### 2,3,4,9-Tetrahydro-1H-carbazol-1-one

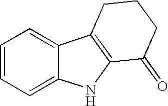

2,3,4,9-Tetrahydro-1H-carbazol-1-one was prepared from aniline and 2-(hydroxymethylene)cyclohexanone in a similar manner as described in Example 1 to give a brown solid. ¹H-NMR (DMSO-d₆): δ 11.6 (s, 1H), 7.66 (d, 1H), 7.38 (d, 1H), 7.30 (t, 1H), 7.07 (t, 1H), 2.90 (t, 2H), 2.56 (t, 2H), 2.15 (quint, 2H); MS m/z 186 (M+1).

### Example 8

### 2,3,4,9-tetrahydro-1H-carbazol-1-amine Hydrochloride

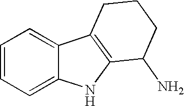

To a solution of 2,3,4,9-tetrahydro-1H-carbazol-1-one (1.5 g, 8.10 mmol) in ethanol (20 mL) was added a solution of hydroxylamine hydrochloride (1.13 g, 16.2 mmol) in water (10 mL) and a solution of sodium acetate (2.19 g, 26.7 mmol) in water (10 mL). The reaction mixture was heated at reflux for 2 h, cooled, and concentrated. The residue was diluted with water and extracted with ethyl acetate (2×100 mL). The organic phase was dried over sodium sulfate, filtered, and concentrated to a brown solid. The oxime was dissolved in THF (80 mL) and lithium aluminum hydride (1.0 M in THF, 24.3 mL) was added dropwise. The reaction was heated at reflux for 7 h and cooled in an ice bath. Methanol was added dropwise until bubbling ceased. The mixture was diluted with aqueous Na/K tartrate, stirred vigorously for 15 min and extracted with ethyl acetate (2×100 mL). The extracts were combined, dried over sodium sulfate, filtered and concentrated. The crude amine was purified by flash chromatography on silica (2% to 5% methanol/methylene chloride gradient) to provide 2,3,4,9-tetrahydro-1H-carbazol-1-amine as a brown oil. The oil was diluted in diethyl ether and HCl (1.0 M in diethyl ether) was added. The resulting precipitate was collected by filtration to provide 2,3,4,9-tetrahydro-1H-carbazol-1-amine hydrochloride (760 mg, 42%) as a light brown solid. ¹H-NMR (CD₃OD): δ 7.54 (d, 1H), 7.42 (d, 1H), 7.22 (t, 1H), 7.09 (t, 1H), 4.66 (t, 1H), 2.95-2.73 (m, 2H), 2.39-2.28 (m, 1H), 2.18-2.03 (m, 3H); MS m/z (M+1) 170.

### Example 9

### N-(6-Bromo-2,3,4,9-tetrahydro-1H-carbazol-1-yl)-N′-phenylurea

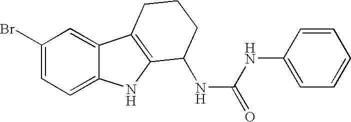

To a solution of 6-bromo-2,3,4,9-tetrahydro-1H-carbazol-1-amine (50 mg, 0.19 mmol) in dichloromethane (1 mL) was added phenyl isocyanate (23 μL, 0.21 mmol). The mixture was stirred at room temperature 15 hours and the resulting precipitate collected by filtration to give a gray solid (62% yield). ¹H-NMR (DMSO-d₆): δ 11.01 (s, 1H), 7.60 (d, 1H), 7.45 (m, 2H), 7.33-7.23 (m, 4H), 7.17 (dd, 1H), 6.94 (m, 1H), 6.63 (d, 1H), 5.02 (m, 1H), 2.68 (m, 2H), 2.06 (m, 1H), 1.95-1.70 (m, 3H); MS m/z 384 (M−1).

### Example 10

### N-(6-Bromo-2,3,4,9-tetrahydro-1H-carbazol-1-yl)-N′-(4-methoxyphenyl)urea

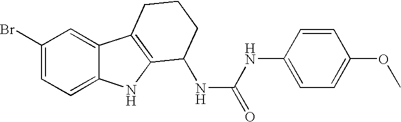

N-(6-Bromo-2,3,4,9-tetrahydro-1H-carbazol-1-yl)-N′-(4-methoxyphenyl)urea was prepared from 6-bromo-2,3,4,9-tetrahydro-1H-carbazol-1-amine and 4-methoxyphenyl isocyanate in a similar manner as described above to give a gray solid (61% yield). ¹H-NMR (DMSO-d₆): δ 10.99 (s, 1H), 8.16 (s, 1H), 7.60 (d, 1H), 7.38-7.27 (m, 3H), 7.17 (dd, 1H), 6.87 (d, 2H), 6.52 (d, 1H), 5.01 (m, 1H), 3.73 (s, 3H), 2.67 (m, 2H), 2.04 (m, 1H), 1.94-1.76 (m, 3H); MS m/z 414 (M−1).

### Example 11

### N-(6-Bromo-2,3,4,9-tetrahydro-1H-carbazol-1-yl)-N′-(4-methoxy-2-methylphenyl)urea

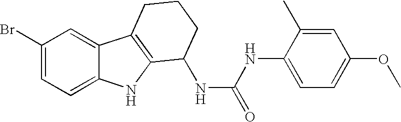

N-(6-Bromo-2,3,4,9-tetrahydro-1H-carbazol-1-yl)-N′-(4-methoxy-2-methylphenyl)urea was prepared from 6-bromo-2,3,4,9-tetrahydro-1H-carbazol-1-amine and 4-methoxy-2-methyl isocyanate in a similar manner as described above to give a dark brown solid (59% yield). ¹H-NMR (DMSO-de): δ 11.00 (s, 1H), 7.71 (d, 1H), 7.60 (d, 1H), 7.47 (s, 1H), 7.32 (d, 1H), 7.17 (dd, 1H), 6.88 (d, 1H), 6.75 (m, 2H), 5.00 (m, 1H), 3.73 (s, 3H), 2.69 (m, 2H), 2.18 (s, 3H), 2.04 (m, 1H), 1.94-1.77 (m, 3H); MS m/z 430 (M+1).

### Example 12

### N-(6-Bromo-2,3,4,9-tetrahydro-1H-carbazol-1-yl)-N′-(3-chloro-4-methoxyphenyl)urea

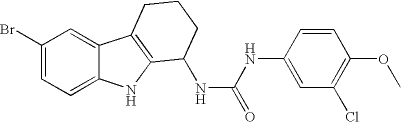

N-(6-Bromo-2,3,4,9-tetrahydro-1H-carbazol-1-yl)-N′-(3-chloro-4-methoxyphenyl)urea was prepared from 6-bromo-2,3,4,9-tetrahydro-1H-carbazol-1-amine and 3-chloro-4-methoxyphenyl isocyanate in a similar manner as described above to give a tan solid (42% yield). ¹H-NMR (DMSO-d₆): δ 10.98 (s, 1H), 8.33 (s, 1H), 7.73 (d, 1H), 7.60 (d, 1H), 7.30 (d, 1H), 7.25-7.14 (m, 2H), 7.08 (d, 1H), 6.63 (d, 1H), 5.01 (m, 1H), 3.82 (s, 3H), 2.65 (m, 2H), 2.04 (m, 1H), 1.94-1.76 (m, 3H); MS m/z 448 (M−1).

### Example 13

### N-(6-Bromo-2,3,4,9-tetrahydro-1H-carbazol-1-yl)-N′-[4-(dimethylamino)phenyl]urea

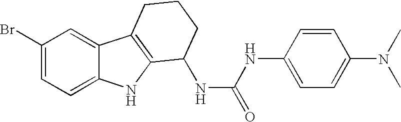

N-(6-Bromo-2,3,4,9-tetrahydro-1H-carbazol-1-yl)-N′-[4-(dimethylamino)phenyl]urea was prepared from 6-bromo-2,3,4,9-tetrahydro-1H-carbazol-1-amine and 4-dimethylamino phenyl isocyanate in a similar manner as described above to give a tan solid (40% yield). ¹H-NMR (DMSO-d₆): δ 10.99 (s, 1H), 7.98 (s, 1H), 7.60 (d, 1H), 7.33-7.23 (m, 3H), 7.17 (dd, 1H), 6.71 (d, 2H), 6.44 (d, 1H), 5.00 (m, 1H), 2.84 (s, 6H), 2.67 (m, 2H), 2.04 (m, 1H), 1.94-1.74 (m, 3H); MS m/z 427 (M−1).

### Example 14A

### N-(6-Bromo-2,3,4,9-tetrahydro-1H-carbazol-1-yl)benzamide

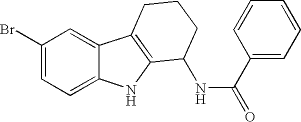

To a solution of 6-bromo-2,3,4,9-tetrahydro-1H-carbazol-1-amine (50 mg, 0.19 mmol) and benzoyl chloride (24 μL, 0.21 mmol) in dichloromethane (1 mL) at 0° C. was added diisopropylethylamine (66 μL, 0.38 mmol). The mixture was stirred at room temperature 15 hours, the solvent removed, and the residue purified by flash chromatography (0-30% ethyl acetate-hexanes) to give 18 mg (26% yield) of a yellow solid. ¹H-NMR (CDCl₃): δ 8.97 (s, 1H), 7.78 (d, 2H), 7.61 (m, 1H), 7.52 (m, 1H), 7.44 (t, 2H), 7.23 (m, 1H), 7.17 (d, 1H), 6.42 (d, 1H), 5.34 (m, 1H), 2.72 (m, 2H), 2.32 (m, 1H), 2.02-1.93 (m, 3H); MS m/z 369 (M−1).

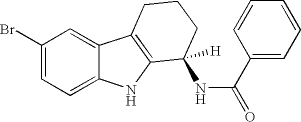

### Example 14B

### N-[(1R)-6-bromo-2,3,4,9-tetrahydro-1H-carbazol-1-yl]benzamide

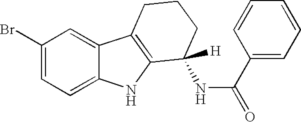

### Example 14C

### N-[(1S)-6-bromo-2,3,4,9-tetrahydro-1H-carbazol-1-yl]benzamide

N-(6-Bromo-2,3,4,9-tetrahydro-1H-carbazol-1-yl)benzamide was separated on a Berger analytical SFC with an HP1100 diode array detector. The sample was monitored at 230 nm under the following conditions: 30% MeOH in CO₂ with a total flow rate of 2 mL/minute at 2250 psi, 50° C. on a Diacel AD-H column (Chiral Technologies), 4.6×250 mm, 5 μm to give N-[(1R)-6-bromo-2,3,4,9-tetrahydro-1H-carbazol-1-yl]benzamide (Example 14B; retention time=12.37 minutes) and N-[(1S)-6-bromo-2,3,4,9-tetrahydro-1H-carbazol-1-yl]benzamide (Example 14C; retention time=16.11 minutes). Chirality assigned by VCD spectroscopy.

### Example 15

### N-(6-Bromo-2,3,4,9-tetrahydro-1H-carbazol-1-yl)-2-phenylacetamide

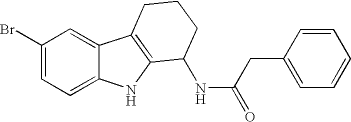

N-(6-Bromo-2,3,4,9-tetrahydro-1H-carbazol-1-yl)-2-phenylacetamide was prepared from 6-bromo-2,3,4,9-tetrahydro-1H-carbazol-1-amine and phenyl acetyl chloride in a similar manner as described above to give a white solid (86% yield). ¹H-NMR (CDCl₃): δ 8.88 (s, 1H), 7.57 (m, 1H), 7.37-7.27 (m, 3H), 7.27-7.20 (m, 3H), 7.15 (d, 1H), 5.74 (d, 1H), 5.08 (m, 1H), 3.61 (s, 2H), 2.62 (m, 2H), 2.13 (m, 1H), 1.81 (m, 2H), 1.67 (m, 1H); MS m/z 383 (M−1).

### Example 16

### N-(6-Bromo-2,3,4,9-tetrahydro-1H-carbazol-1-yl)-3-phenylpropanamide

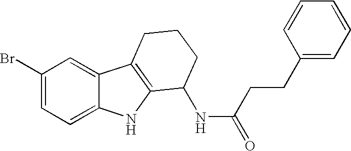

N-(6-Bromo-2,3,4,9-tetrahydro-1H-carbazol-1-yl)-3-phenylpropanamide was prepared from 6-bromo-2,3,4,9-tetrahydro-1H-carbazol-1-amine and hydrocinnamoyl chloride in a similar manner as described above to give a white solid (53% yield). ¹H-NMR (CDCl₃): δ 8.43 (s, 1H), 7.58 (m, 1H), 7.28-7.21 (m, 5H), 7.20-7.15 (m, 2H), 5.51 (d, 1H), 5.05 (m, 1H), 3.00 (t, 2H), 2.62 (m, 2H), 2.58-2.43 (m, 2H), 2.12 (m, 1H), 1.80 (m, 2H), 1.67 (m, 1H); MS m/z 397 (M−1).

### Example 17

### N-(6-Bromo-2,3,4,9-tetrahydro-1H-carbazol-1-yl)-3-phenylprop-2-enamide

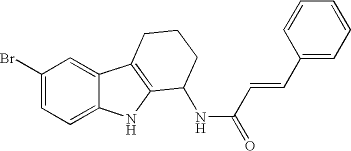

N-(6-Bromo-2,3,4,9-tetrahydro-1H-carbazol-1-yl)-3-phenylprop-2-enamide was prepared from 6-bromo-2,3,4,9-tetrahydro-1H-carbazol-1-amine and cinnamoyl chloride in a similar manner as described above to give an off-white solid (35% yield). ¹H-NMR (CDCl₃): δ 8.98 (s, 1H), 7.69 (d, 1H), 7.60 (m, 1H), 7.48 (m, 2H), 7.36 (m, 3H), 7.22 (dd, 1H), 7.17 (d, 1H), 6.41 (d, 1H), 5.98 (d, 1H), 5.25 (m, 1H), 2.68 (m, 2H), 2.26 (m, 1H), 1.99-1.84 (m, 3H); MS m/z 395 (M−1).

### Example 18

### Benzyl 6-bromo-2,3,4,9-tetrahydro-1H-carbazol-1-ylcarbamate

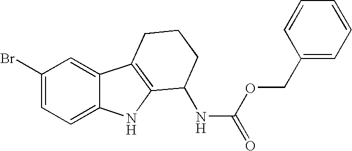

Benzyl 6-bromo-2,3,4,9-tetrahydro-1H-carbazol-1-ylcarbamate was prepared from 6-bromo-2,3,4,9-tetrahydro-1H-carbazol-1-amine and benzyl chloroformate in a similar manner as described above to give a white solid (16% yield). ¹H-NMR (CDCl₃): δ 8.69 (s, 1H), 7.60 (s, 1H), 7.43-7.31 (m, 5H), 7.27-7.21 (m, 1H), 7.17 (d, 1H), 5.16 (q, 2H), 5.08 (m, 1H), 4.92 (m, 1H), 2.66 (m, 2H), 2.20 (m, 1H), 1.89 (m, 2H), 1.80 (m, 1H); MS m/z 400 (M+1).

### Example 19

### N-(6-Bromo-2,3,4,9-tetrahydro-1H-carbazol-1-yl)-2,6-dichlorobenzamide

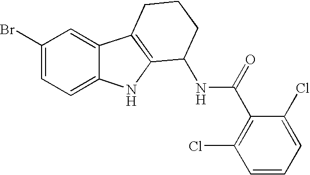

N-(6-Bromo-2,3,4,9-tetrahydro-1H-carbazol-1-yl)-2,6-dichlorobenzamide was prepared from 6-bromo-2,3,4,9-tetrahydro-1H-carbazol-1-amine and 2,6-dichlorobenzoyl chloride in a similar manner as described above to give a white solid (25% yield). ¹H-NMR (DMSO-d₆): δ 10.80 (s, 1H), 9.18 (d, 1H), 7.61 (d, 1H), 7.52 (d, 1H), 7.49 (d, 1H), 7.43 (dd, 1H), 7.36 (d, 1H), 7.19 (dd, 1H), 5.27 (m, 1H), 2.69 (m, 2H), 2.08-1.82 (m, 4H); MS m/z 437 (M−1).

### Example 20

### N-(6-Bromo-2,3,4,9-tetrahydro-1H-carbazol-1-yl)-4-fluorobenzamide

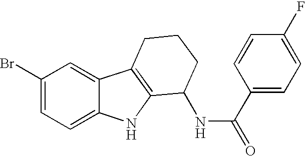

N-(6-Bromo-2,3,4,9-tetrahydro-1H-carbazol-1-yl)-4-fluorobenzamide was prepared from 6-bromo-2,3,4,9-tetrahydro-1H-carbazol-1-amine and 4-fluorobenzoyl chloride in a similar manner as described above to give a white solid (15% yield). ¹H-NMR (DMSO-d₆): δ 11.03 (s, 1H), 8.90 (d, 1H), 8.05 (m, 2H), 7.61 (d, 1H), 7.34 (d, 2H), 7.30 (s, 1H), 7.17 (dd, 1H), 5.38 (m, 1H), 2.68 (m, 2H), 2.09 (m, 2H), 1.86 (m, 2H); MS m/z 387 (M−1).

### Example 21

### N-(6-Bromo-2,3,4,9-tetrahydro-1H-carbazol-1-yl)-4-methoxybenzamide

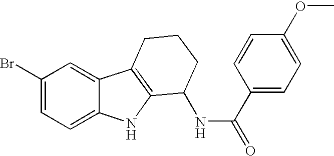

N-(6-Bromo-2,3,4,9-tetrahydro-1H-carbazol-1-yl)-4-methoxybenzamide was prepared from 6-bromo-2,3,4,9-tetrahydro-1H-carbazol-1-amine and p-anisoyl chloride in a similar manner as described above to give a white solid (11% yield). ¹H-NMR (DMSO-d₆): δ 10.99 (s, 1H), 8.70 (d, 1H), 7.96 (d, 2H), 7.60 (d, 1H), 7.29 (d, 1H), 7.16 (dd, 1H), 7.02 (d, 2H), 5.38 (m, 1H), 2.67 (m, 2H), 2.08 (m, 2H), 1.87 (m, 2H); MS m/z 399 (M−1).

### Example 22

### N-(6-Bromo-2,3,4,9-tetrahydro-1H-carbazol-1-yl)-4-nitrobenzamide

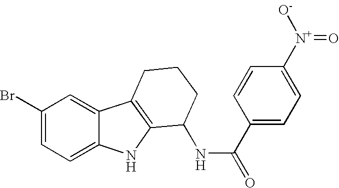

N-(6-Bromo-2,3,4,9-tetrahydro-1H-carbazol-1-yl)-4-nitrobenzamide was prepared from 6-bromo-2,3,4,9-tetrahydro-1H-carbazol-1-amine and 4-nitrobenzoyl chloride in a similar manner as described above to give an orange solid (32% yield). ¹H-NMR (DMSO-d₆): δ 11.11 (s, 1H), 9.24 (d, 1H), 8.36 (d, 2H), 8.19 (d, 2H), 7.62 (m, 1H), 7.29 (d, 1H), 7.18 (dd, 1H), 5.41 (m, 1H), 2.69 (m, 2H), 2.10 (m, 2H), 1.89 (m, 2H); MS m/z 414 (M−1).

### Example 23

### N-(6-Bromo-2,3,4,9-tetrahydro-1H-carbazol-1-yl)-4-chlorobenzamide

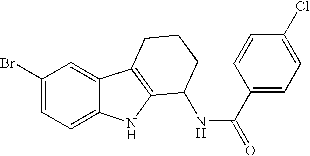

N-(6-Bromo-2,3,4,9-tetrahydro-1H-carbazol-1-yl)-4-chlorobenzamide was prepared from 6-bromo-2,3,4,9-tetrahydro-1H-carbazol-1-amine and 4-chlorobenzoyl chloride in a similar manner as described above to give a tan solid (69% yield). ¹H-NMR (CDCl₃): δ 8.89 (s, 1H), 7.71 (d, 2H), 7.60 (m, 1H), 7.40 (d, 2H), 7.23 (dd, 1H), 7.17 (dd, 1H), 6.36 (d, 1H), 5.30 (m, 1H), 2.69 (m, 2H), 2.29 (m, 1H), 1.96 (m, 3H); MS m/z 403 (M−1).

### Example 24

### N-(6-Bromo-2,3,4,9-tetrahydro-1H-carbazol-1-yl)-4-methylbenzamide

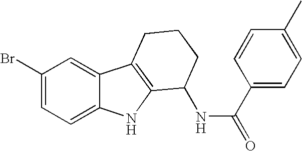

N-(6-Bromo-2,3,4,9-tetrahydro-1H-carbazol-1-yl)-4-methylbenzamide was prepared from 6-bromo-2,3,4,9-tetrahydro-1H-carbazol-1-amine and p-toluoyl chloride in a similar manner as described above to give a yellow solid (62% yield). ¹H-NMR (CDCl₃): δ 8.97 (s, 1H), 7.67 (d, 2H), 7.61 (m, 1H), 7.25-7.20 (m, 3H), 7.17 (d, 1H), 6.36 (d, 1H), 5.30 (m, 1H), 2.71 (m, 2H), 2.39 (s, 3H), 2.30 (m, 1H), 1.97 (m, 3H); MS m/z 383 (M−1).

### Example 25

### N-(6-Bromo-2,3,4,9-tetrahydro-1H-carbazol-1-yl)-4-(trifluoromethyl)benzamide

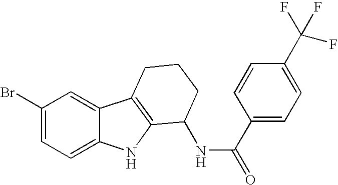

N-(6-Bromo-2,3,4,9-tetrahydro-1H-carbazol-1-yl)-4-(trifluoromethyl)benzamide was prepared from 6-bromo-2,3,4,9-tetrahydro-1H-carbazol-1-amine and 4-trifluoromethyl benzoyl chloride in a similar manner as described above to give a tan solid (63% yield). ¹H-NMR (CDCl₃): δ 8.83 (s, 1H), 7.89 (d, 2H), 7.70 (d, 2H), 7.61 (m, 1H), 7.23 (d, 1H), 7.18 (d, 1H), 6.42 (d, 1H), 5.33 (m, 1H), 2.72 (m, 2H), 2.31 (m, 1H), 1.97 (m, 3H); MS m/z 437 (M−1).

### Example 26

### N-(6-Bromo-2,3,4,9-tetrahydro-1H-carbazol-1-yl)-3-fluorobenzamide

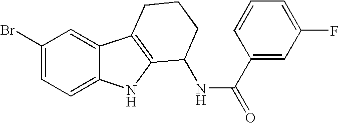

N-(6-Bromo-2,3,4,9-tetrahydro-1H-carbazol-1-yl)-3-fluorobenzamide was prepared from 6-bromo-2,3,4,9-tetrahydro-1H-carbazol-1-amine and 3-fluorobenzoyl chloride in a similar manner as described above to give a white solid (24% yield). ¹H-NMR (DMSO-d₆): δ 11.04 (s, 1H), 8.97 (d, 1H), 7.85-7.81 (m, 1H), 7.81-7.74 (m, 1H), 7.62 (d, 1H), 7.59-7.50 (m, 1H), 7.46-7.37 (m, 1H), 7.29 (d, 1H), 7.17 (dd, 1H), 5.38 (m, 1H), 2.68 (m, 2H), 2.08 (m, 2H), 1.87 (m, 2H); MS m/z 387 (M−1).

### Example 27

### N-(6-Bromo-2,3,4,9-tetrahydro-1H-carbazol-1-yl)-3-methoxybenzamide

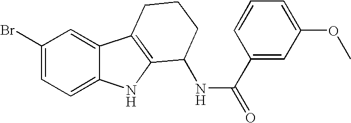

N-(6-Bromo-2,3,4,9-tetrahydro-1H-carbazol-1-yl)-3-methoxybenzamide was prepared from 6-bromo-2,3,4,9-tetrahydro-1H-carbazol-1-amine and m-anisoyl chloride in a similar manner as described above to give an orange solid (49% yield). ¹H-NMR (CDCl₃): δ 8.92 (s, 1H), 7.61 (m, 1H), 7.38-7.30 (m, 2H), 7.30-7.25 (m, 1H), 7.23 (m, 1H), 7.17 (d, 1H), 7.08-7.02 (m, 1H), 6.39 (d, 1H), 5.32 (m, 1H), 3.85 (s, 3H), 2.72 (m, 2H), 2.31 (m, 1H), 1.97 (m, 3H); MS m/z 399 (M−1).

### Example 28

### N-(6-Bromo-2,3,4,9-tetrahydro-1H-carbazol-1-yl)-3-methylbenzamide

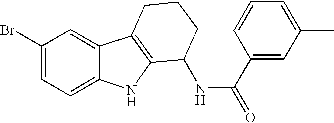

N-(6-Bromo-2,3,4,9-tetrahydro-1H-carbazol-1-yl)-3-methylbenzamide was prepared from 6-bromo-2,3,4,9-tetrahydro-1H-carbazol-1-amine and m-toluoyl chloride in a similar manner as described above to give a tan solid (51% yield). ¹H-NMR (CDCl₃): δ 8.95 (s, 1H), 7.63-7.59 (m, 2H), 7.55 (m, 1H), 7.33 (m, 2H), 7.23 (m, 1H), 7.17 (d, 1H), 6.38 (d, 1H), 5.32 (m, 1H), 2.72 (m, 2H), 2.40 (s, 3H), 2.31 (m, 1H), 1.98 (m, 3H); MS m/z 383 (M−1).

### Example 29

### N-(6-Bromo-2,3,4,9-tetrahydro-1H-carbazol-1-yl)-2-fluorobenzamide

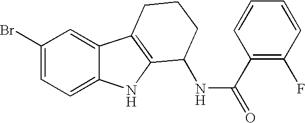

N-(6-Bromo-2,3,4,9-tetrahydro-1H-carbazol-1-yl)-2-fluorobenzamide was prepared from 6-bromo-2,3,4,9-tetrahydro-1H-carbazol-1-amine and 2-fluorobenzoyl chloride in a similar manner as described above to give a yellow solid (57% yield). ¹H-NMR (CDCl₃): δ 8.91 (s, 1H), 8.15 (m, 1H), 7.62 (m, 1H), 7.50 (m, 1H), 7.30 (m, 1H), 7.23 (dd, 1H), 7.18 (d, 1H), 7.12 (m, 2H), 5.35 (m, 1H), 2.72 (m, 2H), 2.31 (m, 1H), 1.98 (m, 3H); MS m/z 387 (M−1).

### Example 30

### N-(6-Bromo-2,3,4,9-tetrahydro-1H-carbazol-1-yl)-2-methoxybenzamide

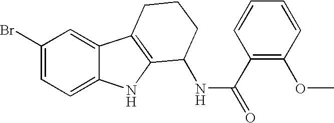

N-(6-Bromo-2,3,4,9-tetrahydro-1H-carbazol-1-yl)-2-methoxybenzamide was prepared from 6-bromo-2,3,4,9-tetrahydro-1H-carbazol-1-amine and oanisoyl chloride in a similar manner as described above to give a pale orange solid (67% yield). ¹H-NMR (CDCl₃): δ 9.06 (s, 1H), 8.31 (d, 1H), 8.25 (dd, 1H), 7.60 (d, 1H), 7.46 (m, 1H), 7.20 (dd, 1H), 7.17 (dd, 1H), 7.10 (m, 1H), 6.96 (d, 1H), 5.31 (m, 1H), 3.91 (s, 3H), 2.72 (m, 2H), 2.31 (m, 1H), 1.98 (m, 3H); MS m/z 399 (M−1).

### Example 31

### N-(6-Bromo-2,3,4,9-tetrahydro-1H-carbazol-1-yl)-2-nitrobenzamide

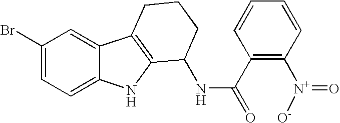

N-(6-Bromo-2,3,4,9-tetrahydro-1H-carbazol-1-yl)-2-nitrobenzamide was prepared from 6-bromo-2,3,4,9-tetrahydro-1H-carbazol-1-amine and 2-nitrobenzoyl chloride in a similar manner as described above to give an orange solid (39% yield). ¹H-NMR (CDCl₃): δ 8.73 (s, 1H), 8.08 (m, 1H), 7.68 (m, 1H), 7.60 (m, 2H), 7.54 (m, 1H), 7.29-7.21 (m, 2H), 6.15 (d, 1H), 5.42 (m, 1H), 2.69 (m, 2H), 2.33 (m, 1H), 1.96 (m, 3H); MS m/z 414 (M−1).

### Example 32

### N-(6-Bromo-2,3,4,9-tetrahydro-1H-carbazol-1-yl)-2-chlorobenzamide

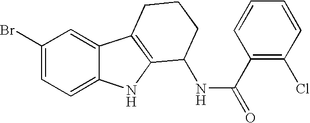

N-(6-Bromo-2,3,4,9-tetrahydro-1H-carbazol-1-yl)-2-chlorobenzamide was prepared from 6-bromo-2,3,4,9-tetrahydro-1H-carbazol-1-amine and 2-chlorobenzoyl chloride in a similar manner as described above to give a white solid (30% yield). ¹H-NMR (DMSO-d₆): δ 10.98 (s, 1H), 8.89 (d, 1H), 7.54 (dd, 2H), 7.48-7.44 (m, 1H), 7.42 (dd, 1H), 7.40-7.34 (m, 1H), 7.28 (d, 1H), 7.13 (dd, 1H), 5.26 (m, 1H), 2.60 (m, 2H), 2.08-1.91 (m, 2H), 1.90-1.73 (m, 2H); MS m/z 403 (M−1).

### Example 33

### N-(6-Bromo-2,3,4,9-tetrahydro-1H-carbazol-1-yl)-2-methylbenzamide

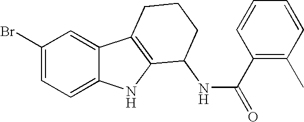

N-(6-Bromo-2,3,4,9-tetrahydro-1H-carbazol-1-yl)-2-methylbenzamide was prepared from 6-bromo-2,3,4,9-tetrahydro-1H-carbazol-1-amine and o-toluoyl chloride in a similar manner as described above to give a gray solid (19% yield). ¹H-NMR (DMSO-d₆): δ 11.01 (s, 1H), 8.70 (d, 1H), 7.59 (m, 1H), 7.47 (d, 1H), 7.33 (m, 2H), 7.26 (m, 2H), 7.17 (dd, 1H), 5.34 (m, 1H), 2.65 (m, 2H), 2.42 (s, 3H), 2.07 (m, 2H), 1.87 (m, 2H); MS m/z 383 (M−1).

### Example 34

### N-(2,3,4,9-Tetrahydro-1H-carbazol-1-yl)benzamide

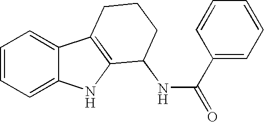

N-(2,3,4,9-Tetrahydro-1H-carbazol-1-yl)benzamide was prepared from 2,3,4,9-tetrahydro-1H-carbazol-1-amine hydrochloride and benzoyl chloride in a similar manner as described above to give a pale yellow solid (73% yield). ¹H-NMR (CDCl₃): δ 8.79 (s, 1H), 7.81-7.75 (m, 2H), 7.54-7.48 (m, 2H), 7.47-7.40 (m, 2H), 7.32 (d, 1H), 7.17 (m, 1H), 7.09 (d, 1H), 6.42 (d, 1H), 5.37 (m, 1H), 2.78 (m, 2H), 2.32 (m, 1H), 2.03-1.91 (m, 3H); MS m/z 289 (M−1).

### Example 35

### N-(6-Methyl-2,3,4,9-tetrahydro-1H-carbazol-1-yl)benzamide

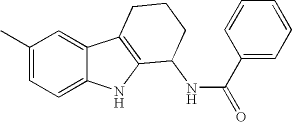

N-(6-Methyl-2,3,4,9-tetrahydro-1H-carbazol-1-yl)benzamide was prepared from 6-methyl-2,3,4,9-tetrahydro-1H-carbazol-1-amine and benzoyl chloride in a similar manner as described above to give a pale yellow solid (81% yield). ¹H-NMR (CDCl₃): δ 8.63 (s, 1H), 7.81-7.75 (m, 2H), 7.54-7.48 (m, 1H), 7.47-7.40 (m, 2H), 7.28 (m, 1H), 7.20 (d, 1H), 6.99 (dd, 1H), 6.40 (d, 1H), 5.36 (m, 1H), 2.75 (m, 2H), 2.44 (s, 3H), 2.31 (m, 1H), 2.02-1.91 (m, 3H); MS m/z 303 (M−1).

### Example 36A

### N-(6-Chloro-2,3,4,9-tetrahydro-1H-carbazol-1-yl)benzamide

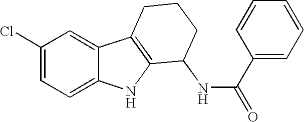

N-(6-Chloro-2,3,4,9-tetrahydro-1H-carbazol-1-yl)benzamide was prepared from 6-chloro-2,3,4,9-tetrahydro-1H-carbazol-1-amine and benzoyl chloride in a similar manner as described above to give a pale yellow solid (81% yield). ¹H-NMR (CDCl₃): δ 9.02 (s, 1H), 7.79-7.73 (m, 2H), 7.54-7.48 (m, 1H), 7.46-7.39 (m, 3H), 7.21 (d, 1H), 7.10 (dd, 1H), 6.45 (d, 1H), 5.32 (m, 1H), 2.72 (m, 2H), 2.30 (m, 1H), 2.01-1.90 (m, 3H); MS m/z 323 (M−1).

### Example 36B

### N-[(1R)-6-chloro-2,3,4,9-tetrahydro-1H-carbazol-1-yl]benzamide

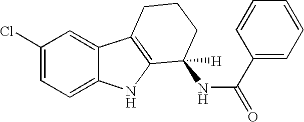

### Example 36C

### N-[(1S)-6-chloro-2,3,4,9-tetrahydro-1H-carbazol-1-yl]benzamide

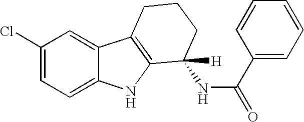

N-(6-Chloro-2,3,4,9-tetrahydro-1H-carbazol-1-yl)benzamide was separated on a Berger analytical SFC with an HP1100 diode array detector. The sample was monitored at 230 nm under the following conditions: 30% MeOH in CO₂ with a total flow rate of 2 mL/minute at 1500 psi, 40° C. on a Diacel AS-H column (Chiral Technologies), 4.6×250 mm, 5 μm to give R-N-(6-chloro-2,3,4,9-tetrahydro-1H-carbazol-1-yl)benzamide (retention time=5.08 minutes) and S-N-(6-chloro-2,3,4,9-tetrahydro-1H-carbazol-1-yl)benzamide (retention time=7.45 minutes). Chirality assigned by VCD spectroscopy.

### Example 37

### N-(6-Bromo-2,3,4,9-tetrahydro-1H-carbazol-1-yl)-4-methylbenzenesulfonamide

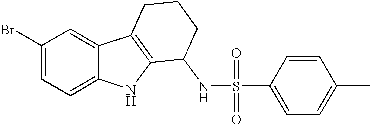

N-(6-Bromo-2,3,4,9-tetrahydro-1H-carbazol-1-yl)-4-methylbenzenesulfonamide was prepared from 6-bromo-2,3,4,9-tetrahydro-1H-carbazol-1-amine and p-toluene sulfonyl chloride in a similar manner as described above to give a tan solid (60% yield). ¹H-NMR (CDCl₃): δ 8.63 (s, 1H), 7.85 (d, 2H), 7.57 (m, 1H), 7.37 (d, 2H), 7.25 (m, 1H), 7.18 (d, 1H), 4.74 (d, 1H), 4.46 (m, 1H), 2.61 (m, 2H), 2.48 (s, 3H), 2.00-1.82 (m, 2H), 1.80-1.60 (m, 2H); MS m/z 419 (M−1).

### Example 38

### N-(6-Bromo-2,3,4,9-tetrahydro-1H-carbazol-1-yl)pyridine-2-carboxamide

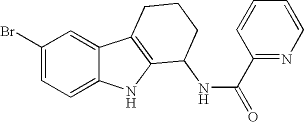

N-(6-Bromo-2,3,4,9-tetrahydro-1H-carbazol-1-yl)pyridine-2-carboxamide was prepared from 6-bromo-2,3,4,9-tetrahydro-1H-carbazol-1-amine and picolinoyl chloride in a similar manner as described above to give an off-white solid (64% yield). ¹H-NMR (CDCl₃): δ 8.96 (s, 1H), 8.52 (m, 1H), 8.37 (d, 1H), 8.21 (m, 1H), 7.86 (m, 1H), 7.61 (m, 1H), 7.43 (m, 1H), 7.22 (dd, 1H), 7.17 (d, 1H), 5.31 (m, 1H), 2.72 (m, 2H), 2.31 (m, 1H), 1.98 (m, 3H); MS m/z 370 (M−1).

### Example 39

### N-(6-Bromo-2,3,4,9-tetrahydro-1H-carbazol-1-yl)nicotinamide

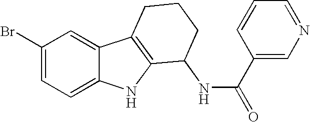

N-(6-Bromo-2,3,4,9-tetrahydro-1H-carbazol-1-yl)nicotinamide was prepared from 6-bromo-2,3,4,9-tetrahydro-1H-carbazol-1-amine and nicotinyl chloride in a similar manner as described above to give a white solid (54% yield). ¹H-NMR (DMSO-d₆): δ 11.02 (s, 1H), 9.08-9.00 (m, 2H), 8.69 (dd, 1H), 8.25 (m, 1H), 7.57 (d, 1H), 7.53-7.43 (m, 1H), 7.24 (d, 1H), 7.13 (dd, 1H), 5.35 (m, 1H), 2.64 (m, 2H), 2.04 (m, 2H), 1.84 (m, 2H); MS m/z 370 (M−1).

### Example 40

### N-(6-Bromo-2,3,4,9-tetrahydro-1H-carbazol-1-yl)-6-chloronicotinamide

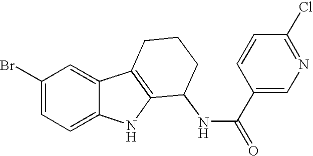

N-(6-Bromo-2,3,4,9-tetrahydro-1H-carbazol-1-yl)-6-chloronicotinamide was prepared from 6-bromo-2,3,4,9-tetrahydro-1H-carbazol-1-amine and 6-chloronicotinyl chloride in a similar manner as described above to give a white solid (48% yield). ¹H-NMR (DMSO-de): δ 11.04 (s, 1H), 9.12 (d, 1H), 8.88 (dd, 1H), 8.30 (dd, 1H), 7.64 (dd, 1H), 7.57 (d, 1H), 7.24 (m, 1H), 7.13 (dd, 1H), 5.34 (m, 1H), 2.63 (m, 2H), 2.04 (m, 2H), 1.82 (m, 2H); MS m/z 404 (M−1).

### Example 41

### N-(6-Bromo-2,3,4,9-tetrahydro-1H-carbazol-1-yl)isonicotinamide

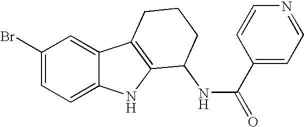

N-(6-Bromo-2,3,4,9-tetrahydro-1H-carbazol-1-yl)isonicotinamide was prepared from 6-bromo-2,3,4,9-tetrahydro-1H-carbazol-1-amine and isonicotinyl chloride in a similar manner as described above to give a white solid (30% yield). ¹H-NMR (DMSO-d₆): δ 11.04 (s, 1H), 9.13 (d, 1H), 8.71 (m, 2H), 7.82 (m, 2H), 7.57 (d, 1H), 7.24 (d, 1H), 7.13 (dd, 1H), 5.35 (m, 1H), 2.64 (m, 2H), 2.04 (m, 2H), 1.84 (m, 2H); MS m/z 370 (M−1).

### Example 42

### N-Phenyl-N′-(2,3,4,9-tetrahydro-1H-carbazol-1-yl)urea

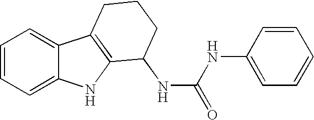

N-Phenyl-N′-(2,3,4,9-tetrahydro-1H-carbazol-1-yl)urea was prepared from 2,3,4,9-tetrahydro-1H-carbazol-1-amine hydrochloride and phenyl isocyanate in a similar manner as described above to give a white solid (60% yield). ¹H-NMR (DMSO-d₆): δ 10.72 (s, 1H), 8.28 (s, 1H), 7.43-7.33 (m, 3H), 7.29-7.17 (m, 3H), 7.00 (m, 1H), 6.94-6.83 (m, 2H), 6.52 (d, 1H), 4.95 (m, 1H), 2.62 (m, 2H), 1.99 (m, 1H), 1.88-1.70 (m, 3H); MS m/z 304 (M−1).

### Example 43

### N-(6-Methyl-2,3,4,9-tetrahydro-1H-carbazol-1-yl)-N′-phenylurea

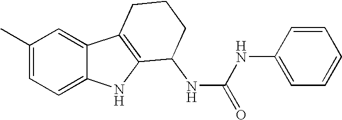

N-(6-Methyl-2,3,4,9-tetrahydro-1H-carbazol-1-yl)-N′-phenylurea was prepared from 6-methyl-2,3,4,9-tetrahydro-1H-carbazol-1-amine and phenyl isocyanate in a similar manner as described above to give a white solid (82% yield). ¹H-NMR (DMSO-d₆): δ 10.57 (s, 1H), 8.27 (s, 1H), 7.42-7.34 (m, 2H), 7.20 (m, 2H), 7.17-7.11 (m, 2H), 6.87 (m, 1H), 6.82 (m, 1H), 6.51 (d, 1H), 4.92 (m, 1H), 2.58 (m, 2H), 2.32 (s, 3H), 1.97 (m, 1H), 1.87-1.70 (m, 3H); MS m/z 318 (M−1).

### Example 44

### N-(6-Chloro-2,3,4,9-tetrahydro-1H-carbazol-1-yl)-N′-phenylurea

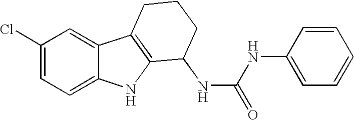

N-(6-Chloro-2,3,4,9-tetrahydro-1H-carbazol-1-yl)N′-phenylurea was prepared from 6-chloro-2,3,4,9-tetrahydro-1H-carbazol-1-amine and phenyl isocyanate in a similar manner as described above to give a white solid (74% yield). ¹H-NMR (DMSO-d₆): δ 10.93 (s, 1H), 8.30 (s, 1H), 7.41-7.36 (m, 3H), 7.28 (d, 1H), 7.21 (m, 2H), 7.00 (dd, 1H), 6.88 (m, 1H), 6.57 (d, 1H), 4.95 (m, 1H), 2.59 (m, 2H), 1.98 (m, 1H), 1.88-1.70 (m, 3H); MS m/z 338 (M−1).

### Example 45A

### N-(6-Chloro-2,3,4,9-tetrahydro-1H-carbazol-1-yl)-2-pyridinecarboxamide

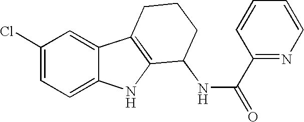

N-(6-Chloro-2,3,4,9-tetrahydro-1H-carbazol-1-yl)-2-pyridinecarboxamide was prepared from 6-chloro-2,3,4,9-tetrahydro-1H-carbazol-1-amine and picolinoyl chloride in a similar manner as described in Example 14 to give an off-white solid (62% yield). ¹H-NMR (DMSO-d₆): δ 10.93 (s, 1H), 8.78 (d, 1H), 8.61 (m, 1H), 8.12 (m, 1H), 8.02 (m, 1H), 7.61 (m, 1H), 7.43 (m, 1H), 7.26 (d, 1H), 7.01 (dd, 1H), 5.34 (m, 1H), 2.64 (m, 2H), 2.00 (m, 3H), 1.82 (m, 1H); MS m/z 348 (M+Na).

### Example 45B

### N-[(1R)-6-chloro-2,3,4,9-tetrahydro-1H-carbazol-1-yl]pyridine-2-carboxamide

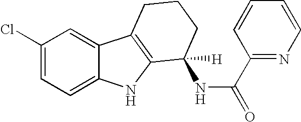

N-(6-Chloro-2,3,4,9-tetrahydro-1H-carbazol-1-yl)-2-pyridinecarboxamide was separated on a Berger analytical SFC with an HP1100 diode array detector. The sample was monitored at 230 nm under the following conditions: 30% MeOH in CO₂ with a total flow rate of 2 mL/minute at 1500 psi, 40° C. on a Diacel AS-H column (Chiral Technologies), 4.6×250 mm, 5 μm to give N-[(1R)-6-chloro-2,3,4,9-tetrahydro-1H-carbazol-1-yl]pyridine-2-carboxamide (retention time=4.55 minutes); [α]_{D}=+86; R configuration confirmed by VCD and X-ray crystallography; ¹H-NMR and MS identical to racemic compound; ¹³C-NMR (DMSO-de): δ 164.1, 150.6, 149.1, 138.4, 136.4, 135.3, 128.5, 127.2, 123.7, 122.7, 121.5, 117.8, 113.4, 111.2, 44.0, 30.8, 21.5, 21.0; HR MS m/z 348.0876 (M+Na); Analytical Calculated for C₁₈H₁₆ClN₃O with ¼H₂O: C, 65.45; H, 5.04; N, 12.72. Found: C, 65.67; H, 4.91; N, 12.66. LC-UV purity check: Waters analytical LC-UV consisting of a Waters 626 pumping system, a Waters 996 diode array detector, and a Gilson 233XL autosampler; column: Waters Symmetry Shield RP18, 3.9×150 mm, 5m; 50-90% acetonitrile-water (0.1% formic acid); total run time of 15 minutes; flow rate remains constant at 1.5mL/minute; retention time=7.41 minutes.

### Example 45C

### N-[(1S)-6-chloro-2,3,4,9-tetrahydro-1H-carbazol-1-yl]pyridine-2-carboxamide

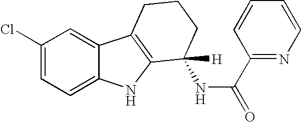

N-(6-Chloro-2,3,4,9-tetrahydro-1H-carbazol-1-yl)-2-pyridinecarboxamide was separated on a Berger analytical SFC with an HP1100 diode array detector. The sample was monitored at 230 nm under the following conditions: 30% MeOH in CO₂ with a total flow rate of 2 mL/minute at 1500 psi, 40° C. on a Diacel AS-H column (Chiral Technologies), 4.6×250 mm, 5 μm to give N-[(1S)-6-chloro-2,3,4,9-tetrahydro-1H-carbazol-1-yl]pyridine-2-carboxamide (retention time=6.77 minutes; [α]_{D}=−86); ¹H-NMR and MS identical to racemic compound. Stereochemistry assigned by VCD.

### Example 46A

### N-(6-Chloro-2,3,4,9-tetrahydro-1H-carbazol-1-yl)-2-fluorobenzamide

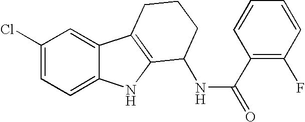

N-(6-Chloro-2,3,4,9-tetrahydro-1H-carbazol-1-yl)-2-fluorobenzamide was prepared from 6-chloro-2,3,4,9-tetrahydro-1H-carbazol-1-amine and 2-fluorobenzoyl chloride in a similar manner as described in Example 14 to give a white solid (63% yield). ¹H-NMR (CDCl₃): δ 8.93 (s, 1H), 8.14 (m, 1H), 7.49 (m, 1H), 7.45 (m, 1H), 7.28 (m, 1H), 7.22 (d, 1H), 7.10 (m, 3H), 5.35 (m, 1H), 2.72 (m, 2H), 2.31 (m, 1H), 1.98 (m, 3H); MS m/z 343 (M+1).

### Example 46B

### N-[(1R)-6-chloro-2,3,4,9-tetrahydro-1H-carbazol-1-yl]-2-fluorobenzamide

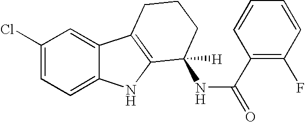

### Example 46C

### N-[(1S)-6-chloro-2,3,4,9-tetrahydro-1H-carbazol-1-yl]-2-fluorobenzamide

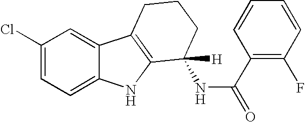

N-(6-Chloro-2,3,4,9-tetrahydro-1H-carbazol-1-yl)-2-fluorobenzamide was separated on a Berger analytical SFC with an HP1100 diode array detector. The sample was monitored at 254 nm under the following conditions: 30% MeOH in CO₂ with a total flow rate of 2 mL/minute at 2250 psi, 40° C. on a Diacel OJ-H column (Chiral Technologies), 4.6×250 mm, 5 μm to give N-[(1S)-6-chloro-2,3,4,9-tetrahydro-1H-carbazol-1-yl]-2-fluorobenzamide (retention time=6.29 minutes) and N-[(1R)-6-chloro-2,3,4,9-tetrahydro-1H-carbazol-1-yl]-2-fluorobenzamide (retention time=9.32 minutes).

### Example 47

### N-(6-Chloro-2,3,4,9-tetrahydro-1H-carbazol-1-yl)-1-methyl-1H-imidazole-5-carboxamide

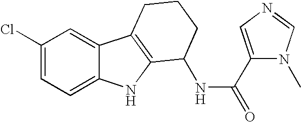

To a solution of 1-methyl-1H-imidazole-5-carboxylic acid (45 mg, 0.36 mmol) in dichloromethane (3.6 mL) was added 6-chloro-2,3,4,9-tetrahydro-1H-carbazol-1-amine (95 mg, 0.43 mmol), 1-ethyl-3-(3-dimethylaminopropyl)carbodiimide (83 mg, 0.43 mmol), and 1-hydroxybenzotriazole (54 mg, 0.40 mmol). After 5 minutes, triethylamine (100 μL, 0.72 mmol) was added and the reaction was stirred at room temperature for 15 hours. The reaction mixture was diluted with dichloromethane, washed with water, 1 N hydrochloric acid, 1 N sodium hydroxide, brine, dried with magnesium sulfate, filtered, and concentrated. The residue was purified by preparative chromatography (10-90% acetonitrile-water (0.1% trifluoroacetic acid)) and then diluted with ethyl acetate, washed with saturated aqueous sodium bicarbonate, and dried with magnesium sulfate to give 43 mg (36% yield) of a white solid. ¹H-NMR (CDCl₃): δ 9.21 (s, 1H), 7.43 (m, 1H), 7.32 (s, 1H), 7.30 (s, 1H), 7.20 (d, 1H), 7.07 (dd, 1H), 6.60 (d, 1H), 5.25 (m, 1H), 3.81 (s, 3H), 2.67 (m, 2H), 2.21 (m, 1H), 1.90 (m, 3H); MS m/z 327 (M−1).

### Example 48

### N-(6-Chloro-2,3,4,9-tetrahydro-1H-carbazol-1-yl)-1-methyl-1H-pyrazole-5-carboxamide

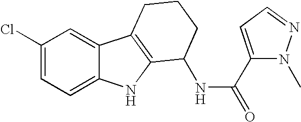

N-(6-Chloro-2,3,4,9-tetrahydro-1H-carbazol-1-yl)-1-methyl-1H-pyrazole-5-carboxamide was prepared from 2-methyl-2H-pyrazole-3-carboxylic acid and 6-chloro-2,3,4,9-tetrahydro-1H-carbazol-1-amine in a similar manner as described above to give a white solid (30% yield). ¹H-NMR (CDCl₃): δ 8.69 (s, 1H), 7.45 (m, 2H), 7.22 (d, 1H), 7.10 (dd, 1H), 6.48 (d, 1H), 6.26 (d, 1H), 5.28 (m, 1H), 4.22 (s, 3H), 2.71 (m, 2H), 2.28 (m, 1H), 1.95 (m, 3H); MS m/z 327 (M−1).

### Example 49

### N-(6-Chloro-2,3,4,9-tetrahydro-1H-carbazol-1-yl)-1-methyl-1H-pyrazole-3-carboxamide

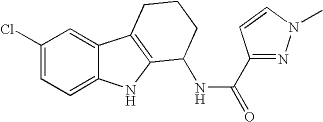

N-(6-Chloro-2,3,4,9-tetrahydro-1H-carbazol-1-yl)-1-methyl-1H-pyrazole-3-carboxamide was prepared from 1-methyl-1H-pyrazole-3-carboxylic acid and 6-chloro-2,3,4,9-tetrahydro-1H-carbazol-1-amine in a similar manner as described above to give an off-white solid (20% yield). ¹H-NMR (CDCl₃): δ 8.95 (s, 1H), 7.43 (m, 1H), 7.37 (m, 1H), 7.22 (d, 1H), 7.07 (dd, 1H), 6.80 (m, 1H), 5.30 (m, 1H), 3.88 (s, 3H), 2.71 (m, 2H), 2.27 (m, 1H), 1.97 (m, 3H); MS m/z 327 (M−1).

### Example 50

### N-(6-Chloro-2,3,4,9-tetrahydro-1H-carbazol-1-yl)-1H-imidazole-4-carboxamide

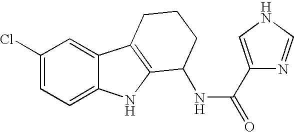

N-(6-Chloro-2,3,4,9-tetrahydro-1H-carbazol-1-yl)-1H-imidazole-4-carboxamide was prepared from 4-imidazole carboxylic acid and 6-chloro-2,3,4,9-tetrahydro-1H-carbazol-1-amine in a similar manner as described above to give a white solid (4% yield). ¹H-NMR (CDCl₃): δ 9.19 (s, 1H), 7.62 (s, 1H), 7.57 (s, 1H), 7.41 (s, 1H), 7.37 (d, 1H), 7.17 (d, 1H), 7.05 (dd, 1H), 6.60 (d, 1H), 5.25 (m, 1H), 2.62 (m, 2H), 2.21 (m, 1H), 1.91 (m, 3H); MS m/z 313 (M−1).

### Example 51

### N-(6-Chloro-2,3,4,9-tetrahydro-1H-carbazol-1-yl)-1H-pyrazole-3-carboxamide

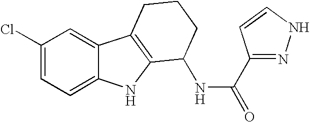

N-(6-Chloro-2,3,4,9-tetrahydro-1H-carbazol-1-yl)-1H-pyrazole-3-carboxamide was prepared from 1H-pyrazole-3-carboxylic acid and 6-chloro-2,3,4,9-tetrahydro-1H-carbazol-1-amine in a similar manner as described above to give a white solid (16% yield). ¹H-NMR (CD₃OD-d₄): δ 7.70 (d, 1H), 7.37 (d, 1H), 7.22 (d, 1H), 6.99 (dd, 1H), 6.81 (d, 1H), 5.37 (m, 1H), 2.70 (m, 2H), 2.19 (m, 1H), 2.00 (m, 3H); MS m/z 313 (M−1).

### Example 52

### N-(6-bromo-2,3,4,9-tetrahydro-1H-carbazol-1-yl)-2,6-difluorobenzamide

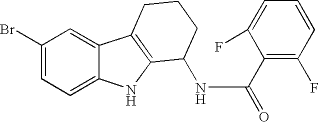

N-(6-bromo-2,3,4,9-tetrahydro-1H-carbazol-1-yl)-2,6-difluorobenzamide was prepared from 2,6-difluorobenzoylchloride and 6-bromo-2,3,4,9-tetrahydro-1H-carbazol-1-amine in a similar manner as described above to give a white solid: ¹H-NMR (CDCl₃): δ 8.80 (s, 1H), 7.60 (m, 1H), 7.35 (m, 1H), 7.23 (overlapped dd and d, 2H), 6.94 (t, 2H), 6.37 (d, 1H), 5.35 (m, 1H), 2.7 (m, 2H), 2.26 (m, 1H), 1.95 (m, 3H); MS m/z 404 (M−1).

### Example 53

### N-(6-bromo-2,3,4,9-tetrahydro-1H-carbazol-1-yl)-2-fluorobenzenesulfonamide

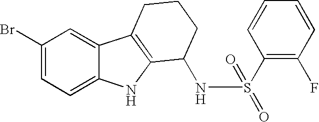

N-(6-bromo-2,3,4,9-tetrahydro-1H-carbazol-1-yl)-2-fluorobenzenesulfonamide was prepared from 2-fluorobenzenesulfonylchloride and 6-bromo-2,3,4,9-tetrahydro-1H-carbazol-1-amine in a similar manner as described above to give a white solid: ¹H-NMR (CDCl₃): δ 8.56 (s, 1H), 8.00 (m, 1H), 7.65 (m, 1H), 7.58 (m, 1H), 7.2-7.4 (m, 3H), 5.04 (d, 1H), 4.60 (m, 1H), 2.65 (m, 2H), 1.6-2.1 (m, 4H); MS m/z 421 (M−1).

### Example 54

### N-(6-bromo-2,3,4,9-tetrahydro-1H-carbazol-1-yl)-2,6-difluorobenzenesulfonamide

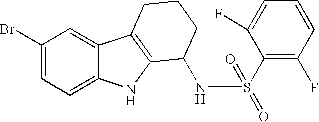

N-(6-bromo-2,3,4,9-tetrahydro-1H-carbazol-1-yl)-2,6-difluorobenzenesulfonamide was prepared from 2,6-difluorobenzenesulfonylchloride and 6-bromo-2,3,4,9-tetrahydro-1H-carbazol-1-amine in a similar manner as described above to give a white solid: ¹H-NMR (CDCl₃): δ 8.60 (s, 1H), 7.48-7.58 (m, 2H), 7.24 (dd, 1H), 7.18 (d, 1H), 7.04 (t, 1H), 5.31 (m, 1H), 4.73 (m, 1H), 2.62 (m, 2H), 1.7-2.1 (m, 4H).

### Example 55

### N-(6-Chloro-2,3,4,9-tetrahydro-1H-carbazol-1-yl)-2-pyrazinecarboxamide

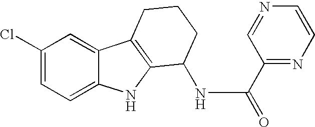

2-piperazine carboxylic acid (0.096 g, 0.77 mmol) was suspended in dichloromethane (2 mL) and oxalyl chloride (0.19 mL, 0.27 g, 2.19 mmol) and DMF (3 drops) were added. The mixture was stirred at ambient temperature for 1 h and then evaporated. The residue was suspended in dichloromethane (3 mL)/DMF (1 mL) and 6-chloro-2,3,4,9-tetrahydro-1H-carbazol-1-amine (0.15 g, 0.68 mmol) was added followed by triethylamine (0.2 mL, 0.147 g, 1.46 mmol). After stirring at ambient temperature for 18 h, water was added and the mixture was extracted with dichloromethane. The organic phase was washed with water and saturated NaHCO₃/water, dried (NaSO₄), concentrated, and chromatographed (Silica gel, EtOAc/hexanes, 0-50%) to provide the title compound as a pale yellow solid (0.092 g, 41% yield). ¹H-NMR (DMSO-d₆): δ 10.91 (s, 1H), 9.24 (s, 1H), 9.02 (d, 1H), 8.85 (d, 1H), 8.7 (s, 1H), 7.42 (s, 1H), 7.25 (d, 1H), 6.99 (d, 1H), 5.4 (m, 1H), 2.61 (m, 2H), 2.0 (m, 3H), 1.8 (m, 1H); MS m/z 327 (M+1).

### Example 56

### N-(6-Chloro-2,3,4,9-tetrahydro-1H-carbazol-1-yl)-6-methyl-2-pyridinecarboxamide

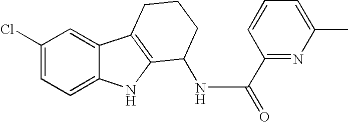

To a solution of 6-chloro-2,3,4,9-tetrahydro-1H-carbazol-1-amine (0.146 g, 0.66 mmol) and 6-methylpicolinic acid (0.1 g, 0.73 mmol) in dichloromethane (2 mL)/DMF (2 mL) under nitrogen atmosphere was added EDCI (0.28 g, 1.46 mmol) and DMAP (0.005 g). After 18 h at ambient temperature, water was added and the mixture was extracted with EtOAc. The organic phase was washed with water, saturated NaHCO₃/water, dried (Na₂SO₄), evaporated, and chromatographed (Silica gel, EtOAc/hexanes 0-40%) to provide the title compound as an off-white solid (0.086 g, 40% yield). ¹H-NMR (DMSO-d₆): δ 10.96 (s, 1H), 8.66 (d, 1H), 7.89 (s, 1H), 7.85 (m, 1H), 7.46 (m, 1H), 7.42 (d, 1H), 7.27 (d, 1H), 7.0 (dd, 1H), 5.33 (quartet, 1H), 2.64 (m, 2H), 2.5 (s 3H), 2.03 (m, 1H), 7.98 (m, 2H), 1.8 (m, 1H); MS m/z 340 (M+1).

### Example 57

### N-(6-Chloro-2,3,4,9-tetrahydro-1H-carbazol-1-yl)-5-isoxazolecarboxamide

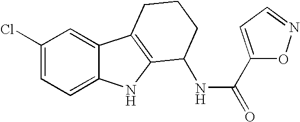

N-(6-Chloro-2,3,4,9-tetrahydro-1H-carbazol-1-yl)-5-isoxazolecarboxamide was prepared from 6-chloro-2,3,4,9-tetrahydro-1H-carbazol-1-amine and isoxazole-5-carbonyl chloride in a similar manner as described above to give a pale green solid (45% yield). ¹H-NMR (DMSO-d₆): δ 11.03 (s, 1H), 9.3 (d, 1H), 8.75 (s, 1H), 7.43 (s, 1H), 7.28 (d, 1H), 7.13 (s, 1H), 7.0 (d, 1H), 5.3 (quartet, 1H), 2.63 (m, 2H), 2.05 (m, 2H), 1.82 (m, 2H); MS m/z 316 (M+1).

### Example 58

### N-(6-Chloro-2,3,4,9-tetrahydro-1H-carbazol-1-yl)-2-furancarboxamide

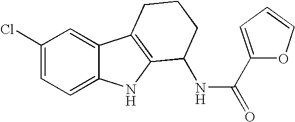

N-(6-Chloro-2,3,4,9-tetrahydro-1H-carbazol-1-yl)-2-furancarboxamide was prepared from 6-chloro-2,3,4,9-tetrahydro-1H-carbazol-1-amine and furoyl chloride in a similar manner as described above to give an off-white solid (55% yield). ¹H-NMR (DMSO-d₆): δ 10.94 (s, 1H), 8.7 (d, 1H), 7.81 (s, 1H), 7.41 (s, 1H), 7.24 (d, 1H), 7.2 (s, 1H), 7.0 (d, 1H), 6.6 (s, 1H), 5.24 (m, 1H), 2.62 (m, 2H), 2.02 (m, 2H), 1.8 (m, 2H); MS m/z 315 (M+1).

### Example 59

### N-(6-Chloro-2,3,4,9-tetrahydro-1H-carbazol-1-yl)-3,5-dimethyl-4-isoxazolecarboxamide

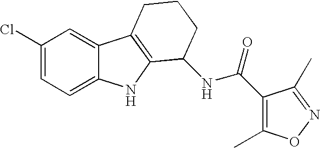

N-(6-Chloro-2,3,4,9-tetrahydro-1H-carbazol-1-yl)-3,5-dimethyl-4-isoxazolecarboxamide was prepared from 6-chloro-2,3,4,9-tetrahydro-1H-carbazol-1-amine and 3,5-dimethylisoxazole-4-carbonyl chloride in a similar manner as described above to give an off-white solid (68% yield). ¹H-NMR (DMSO-d₆): δ 10.99 (s, 1H), 8.4 (d, 1H), 7.44 (s, 1H), 7.15 (d, 1H), 7.0 (d, 1H), 5.14 (quartet, 1H), 2.62 (m, 2H), 2.5 (s, 3H), 2.24 (s, 3H), 2.02 (m, 1H), 1.99 (m, 1H), 1.8 (m, 2H); MS m/z 344 (M+1).

### Example 60

### N-(6-Chloro-2,3,4,9-tetrahydro-1H-carbazol-1-yl)-6-fluoro-2-pyridinecarboxamide

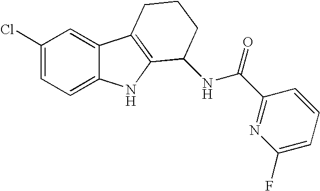

To a solution of 6-chloro-2,3,4,9-tetrahydro-1H-carbazol-1-amine (0.15 g, 0.68 mmol), DIPEA (0.165 mL, 0.123 g, 0.95 mmol) and 2-fluoro-6-pyridine carboxylic acid (0.115 g, 0.816 mmol) in dichloromethane (5 mL) was added O-(1H-benzotriazol-1-yl)-N,N,N′,N′-tetramethyluronium tetrafluoroborate (TBTU, 0.26 g, 0.81 mmol) and the mixture was stirred at ambient temperature for 2 h. Water was added and after 10 min, the organic phase was separated and washed with saturated NaHCO₃/water, dried (Na₂SO₄), evaporated, and chromatographed (Silica gel, EtOAc/hexanes, 0-30%) to provide the title compound as an off-white solid (0.13 g, 61% yield). ¹H-NMR (DMSO-d₆): δ 10.92 (s, 1H), 8.8 (d, 1H), 8.2 (quartet, 1H), 8.04 (dd, 1H), 7.43 (m, 2H), 7.28 (d, 1H), 7.0 (dd, 1H), 5.37 (quartet, 1H), 2.62 (m, 2H), 2.0 (m, 3H), 1.8 (m, 1H); MS m/z 365 (M+Na).

### Example 61

### N-(6-Chloro-2,3,4,9-tetrahydro-1H-carbazol-1-yl)-3-methylpyridine-2-carboxamide

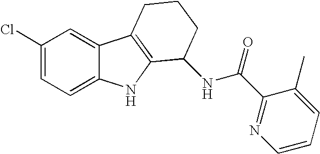

N-(6-Chloro-2,3,4,9-tetrahydro-1H-carbazol-1-yl)-3-methylpyridine-2-carboxamide was prepared from 6-chloro-2,3,4,9-tetrahydro-1H-carbazol-1-amine and 3-methylpicolinic acid in a similar manner as described above to give an off-white solid (55% yield). ¹H-NMR (DMSO-d₆): δ 110.89 (s, 1H), 8.8 (d, 1H), 8.4 (d, 1H), 7.8 (d, 1H), 7.4 (s, 2H), 7.3 (d, 1H), 7.0 (d, 1H), 5.3 (m, 1H), 2.6 (m, 5H), 1.9 (m, 4H); MS m/z 362 (M+Na).

### Example 62

### N-[2-({2-[(2-Aminoethyl)oxy]ethyl}oxy)ethyl]-N′-(6-chloro-2,3,4,9-tetrahydro-1H-carbazol-1-yl)-1,4-benzenedicarboxamide

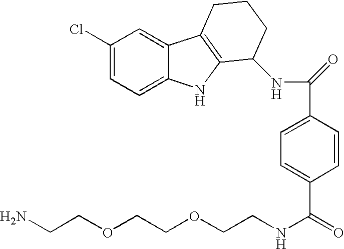

### A): Methyl 4-(14,14-dimethyl-12-oxo-5,8,13-trioxa-2,11-diazapentadecan-1-oyl)benzoate

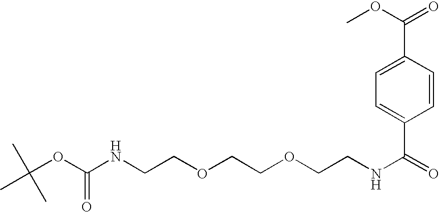

Methyl 4-(14,14-dimethyl-12-oxo-5,8,13-trioxa-2,11-diazapentadecan-1-oyl)benzoate was prepared from 1,1-dimethylethyl [2-({2-[(2-aminoethyl)oxy]ethyl}oxy)ethyl]carbamate (J. Chem. Soc., Dalton Trans., 2000, 1805-1812) and terephthalic acid methyl ester in a similar manner as described above to give a colorless oil (0.25 g, 64% yield). ¹H-NMR (DMSO-d₆): δ 8.7 (t, 1H), 8.0 (d, 2H), 7.95 (d, 2H), 6.75 (t, 1H), 3.82 (s, 3H), 3.4 (m, 10H), 3.0 (m, 2H), 1.35 (s, 9H); MS m/z 411 (M+1).

### B): 4-(14,14-Dimethyl-12-oxo-5,8,13-trioxa-2,11-diazapentadecan-1-oyl)benzoic Acid

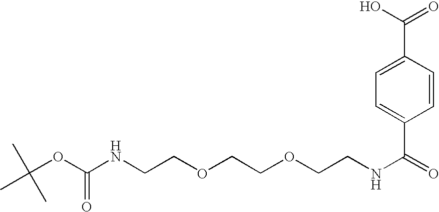

To a solution of methyl_{—}4-(14,14-dimethyl-12-oxo-5,8,13-trioxa-2,11-diazapentadecan-1-oyl)benzoate (0.24, 0.58 mmol) in MeOH/water (3:1, 4 mL) at 0° C. was added lithium hydroxide monohydrate (0.061 g, 1.45b mmol) and the mixture was allowed to warm to ambient temperature in 3.5 h and stirred for 2 h. Water was added and the mixture was acidified to pH˜3 with 1 N HCl/water. The mixture was extracted with Et₂O and the organic phase was dried (Na₂SO₄) and concentrated to give the title compound as an off-white solid (0.20 g, 87% yield). ¹H-NMR (DMSO-d₆): δ 13.17 (s, 1H), 8.7 (t, 1H), 8.0 (d, 2H), 7.9 (d, 2H), 6.7 (t, 1H), 3.4 (m, 10H), 3.0 (m, 2H), 1.39 (s, 9H); MS m/z 397 (M+1).

### C): 1,1-Dimethylethyl [2-({2-[(2-{[(4-{[(6-chloro-2,3,4,9-tetrahydro-1H-carbazol-1-yl)amino]carbonyl}phenyl)carbonyl]amino}ethyl)oxy]ethyl}oxy)ethyl]carbamate

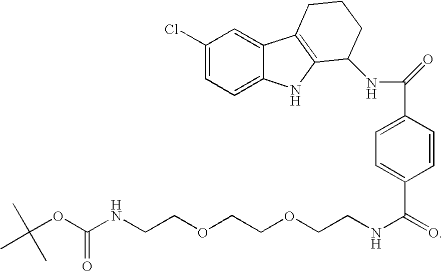

1,1-Dimethylethyl [2-({2-[(2-[(4-{[(6-chloro-2,3,4,9-tetrahydro-1H-carbazol-1-yl)amino]carbonyl}phenyl)carbonyl]amino}ethyl)oxy]ethyl)oxy)ethyl]carbamate was prepared from 6-chloro-2,3,4,9-tetrahydro-1H-carbazol-1-amine and 4-(14,14-dimethyl-12-oxo-5,8,13-trioxa-2,11-diazapentadecan-1-oyl)benzoic acid in a similar manner as described above to provide an off-white solid (58% yield). ¹H-NMR (DMSO-d₆): δ 10.98 (s, 1H), 8.95 (d, 1H), 8.6 (t, 1H), 8.0 (d, 2H), 7.9 (d, 2H), 7.42 (s, 1H), 7.26 (d, 1H), 7.01 (d, 1H), 6.78 (t, 1H), 5.36 (quartet, 1H), 3.4 (m, 10H), 3.0 (quartet, 2H), 2.62 (t, 2H), 2.05 (m, 2H), 1.8 (m, 2H), 1.38 (s, 9H); MS m/z 599 (M+1).

### D): N-[2-({2-[(2-Aminoethyl)oxy]ethyl}oxy)ethyl]-N′-(6-chloro-2,3,4,9-tetrahydro-1H-carbazol-1-yl)-1,4-benzenedicarboxamide

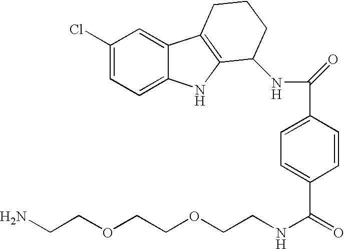

1,1-Dimethylethyl [2-({2-[(2-{[(4-{[(6-chloro-2,3,4,9-tetrahydro-1H-carbazol-1-yl)amino]carbonyl}phenyl)carbonyl]amino}ethyl)oxy]ethyl}oxy)ethyl]carbamate (0.13 g) was dissolved in dichloromethane (4 mL) at 0° C. and TFA (2 mL) was added. After 1 h, solvent was evaporated and the residue was dissolved in dichloromethane, washed with saturated NaHCO₃/water, dried (Na₂SO₄), evaporated, and chromatographed (Silica gel, 2M NH₃ in MeOH/dichloromethane, 0-5%) to give the title compound as a pale yellow solid (0.041 g, 40% yield). ¹H-NMR (DMSO-d₆): δ 10.99 (s, 1H), 8.95 (d, 1H), 8.6 (t, 1H), 7.98 (d, 2H), 7.84 (d, 2H), 7.41 (s, 1H), 7.24 (d, 1H), 7.0 (d, 1H), 5.35 (quartet, 1H), 3.5 (m, 6H), 3.4 (m, 2H), 3.3 (m, 2H), 2.65 (m, 4H), 2.2 (m, 2H), 2.1 (m, 2H), 1.8 (m, 2H); MS m/z 499 (M+1).

### Example 63

### Methyl 6-{[(6-chloro-2,3,4,9-tetrahydro-1H-carbazol-1-yl)amino]carbonyl}-3-pyridinecarboxylate

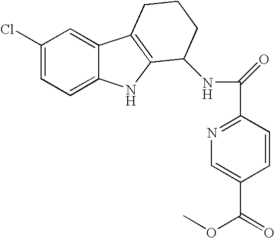

Methyl 6-{[(6-chloro-2,3,4,9-tetrahydro-1H-carbazol-1-yl)amino]carbonyl}-3-pyridinecarboxylate was prepared from 6-chloro-2,3,4,9-tetrahydro-1H-carbazol-1-amine and 5-(methoxycarbonyl)pyridine in a similar manner as described above to give a yellow solid (64%). ¹H-NMR (DMSO-d₆): δ 10.94 (s, 1H), 9.1 (d, 1H), 8.98 (dd, 1H), 8.5 (d, 1H), 8.23 (d, 1H), 7.42 (d, 1H), 7.23 (d, 1H), 7.0 (dd, 1H), 5.35 (quartet, 1H), 3.9 (s, 3H), 2.65 (m, 2H), 2.0 (m, 3H), 1.8 (m, 1H); MS m/z 406 (M+Na).

### Example 64

### 6-{[(6-Chloro-2,3,4,9-tetrahydro-1H-carbazol-1-yl)amino]carbonyl}-3-pyridinecarboxylic Acid

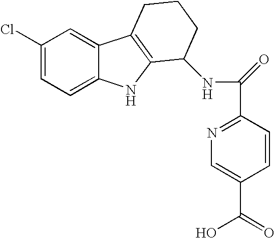

6-{[(6-Chloro-2,3,4,9-tetrahydro-1H-carbazol-1-yl)amino]carbonyl}-3-pyridinecarboxylic acid was prepared from methyl 6-{[(6-chloro-2,3,4,9-tetrahydro-1H-carbazol-1-yl)amino]carbonyl}-3-pyridinecarboxylate in a similar manner as described above to give a yellow solid (98%). ¹H-NMR (DMSO-d₆): δ 13.68 (s, 1H), 10.93 (s, 1H), 9.04 (d, 1H), 8.98 (d, 1H), 8.48 (dd, 1H), 8.22 (d, 1H), 7.42 (d, 1H), 7.23 (d, 1H), 7.0 (dd, 1H), 5.38 (quartet, 1H), 2.63 (m, 2H), 2.0 (m, 3H), 1.8 (m, 1H); MS m/z 370 (M+1).

### Example 65

### 1,1-Dimethylethyl [2-({2-[(2-{[(6-{[(6-chloro-2,3,4,9-tetrahydro-1H-carbazol-1-yl)amino]carbonyl}-3-Pyridinyl)carbonyl]amino}ethyl)oxy]ethyl}oxy)ethyl]carbamate

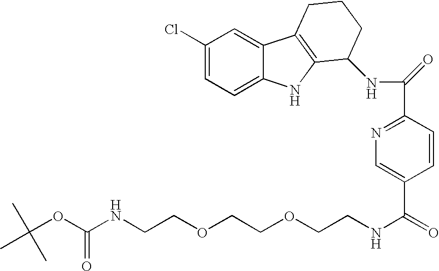

1,1-Dimethylethyl [2-({2-[(2-{[(6-{[(6-chloro-2,3,4,9-tetrahydro-1H-carbazol-1-yl)amino]carbonyl}-3-pyridinyl)carbonyl]amino}ethyl)oxy]ethyl}oxy)ethyl]carbamate was prepared from 6-{[(6-Chloro-2,3,4,9-tetrahydro-1H-carbazol-1-yl)amino]carbonyl}-3-pyridinecarboxylic acid and 1,1-dimethylethyl [2-({2-[(2-aminoethyl)oxy]ethyl}oxy)ethyl]carbamate (J. Chem. Soc., Dalton Trans., 2000, 1805-1812) in a similar manner as described above to provide a yellowish solid foam (59% yield). ¹H-NMR (DMSO-d₆): δ 10.94 (s, 1H), 8.98 (d, 1H), 8.9 (m, 2H), 8.38 (dd, 1H), 8.18 (d, 1H), 7.42 (d, 1H), 7.25 (d, 1H), 7.0 (dd, 1H), 6.73 (t, 1H), 5.34 (quartet, 1H), 3.5 (m, 6H), 3.45 (m, 2H), 3.35 (m, 2H), 3.02 (quartet, 2H), 2.62 (m, 2H), 2.0 (m, 3H), 1.8 (m, 1H), 1.38 (s, 9H); MS m/z 600 (M+1).

### Example 66

### N⁵-[2-({2-[(2-Aminoethyl)oxy]ethyl}oxy)ethyl]-N²-(6-chloro-2,3,4,9-tetrahydro-1H-carbazol-1-yl)-2,5-pyridinedicarboxamide

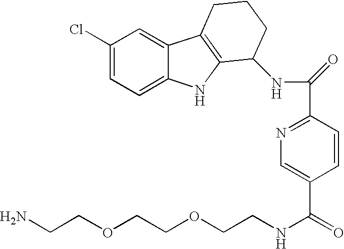

N⁵-[2-({2-[(2-Aminoethyl)oxy]ethyl}oxy)ethyl]-N²-(6-chloro-2,3,4,9-tetrahydro-1H-carbazol-1-yl)-2,5-pyridinedicarboxamide was prepared from 1,1-dimethylethyl [2-({2-[(2-{[(6-{[(6-chloro-2,3,4,9-tetrahydro-1H-carbazol-1-yl)amino]carbonyl}-3-pyridinyl)carbonyl]amino}ethyl)oxy]ethyl}oxy)ethyl]carbamate in a similar manner as described in above to give a light tan solid (90% yield). ¹H-NMR (DMSO-d₆): δ 10.94 (s, 1H), 8.98 (d, 1H), 8.9 (m, 2H), 8.38 (dd, 1H), 8.19 (d, 1H), 7.42 (d, 1H), 7.25 (d, 1H), 7.0 (dd, 1H), 3.5 (m, 10H), 2.6 (m, 4H), 2.0 (m, 3H), 1.8 (m, 2H), 1.5 (m, 2H); MS m/z 500 (M+1).

### Example 67

### N²-(6-Chloro-2,3,4,9-tetrahydro-1H-carbazol-1-yl)-N⁵-(2-{[2-({2-[(phenylcarbonyl)amino]ethyl}oxy)ethyl]oxy}ethyl)-2,5-pyridinedicarboxamide

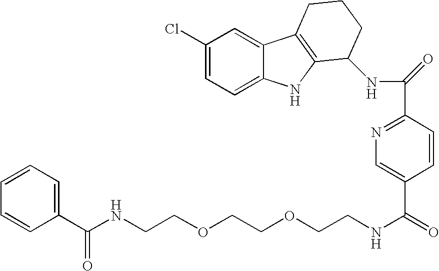

To a solution of N⁵-[2-({2-[(2-aminoethyl)oxy]ethyl}oxy)ethyl]-N²-(6-chloro-2,3,4,9-tetrahydro-1H-carbazol-1-yl)-2,5-pyridinedicarboxamide (0.088 g, 0.18 mmol) in dichloromethane (4 mL) was added triethylamine (0.053 mL, 0.038 g, 0.38 mmol) and benzoyl chloride (0.022 mL, 0.026 g, 0.19 mmol) and the mixture was stirred at ambient temperature for 45 min. The mixture was diluted with dichloromethane and washed with saturated 1 M NaOH/water, dried (Na₂SO₄), evaporated, and chromatographed (Silica gel, MeOH/dichloromethane, 0-5%) to provide the title compound as a yellowish solid foam (0.035 g, 32% yield). ¹H-NMR (DMSO-d₆): δ 10.94 (s, 1H), 8.98 (d, 1H), 8.9 (m, 2H), 8.47 (t, 1H), 8.37 (dd, 1H), 8.17 (d, 1H), 7.8 (d, 2H), 7.5 (m, 4H), 7.24 (d, 1H), 7.0 (dd, 1H), 5.38 (quartet, 1H), 3.52 (m, 8H), 3.4 (m, 4H), 2.62 (m, 2H), 2.0 (m, 3H), 1.8 (m, 1H); MS m/z 605 (M+1).

### Biological Experimentals and Data

Compounds of the current invention are believed useful in the treatment and/or prophylaxis of conditions and diseases associated with HcV infection. Activity mediated through HCV was determined using an ET replicon line.

The Materials used include a Medium comprised of DMEM (1× liquid, high glucose); Invitrogen cat# 11965-092; 100× Penicillin/Streptomycin solution (10,000 Units/mL); Invitrogen cat# 15140-122; 100× Non-essential amino acid solution (10 mM); Invitrogen cat# 11140-050; Fetal bovine serum; JRH Biosciences cat# 12107-500M; Geneticin (50 mg/mL); Invitrogen cat# 10131-035

The Luciferase assay reagents include Steady-Glo Luciferase assay system, cat# E2550 (Promega)

The ET replicon line (see Lohmann et al. (1999), Replication of subgenomic hepatitis C virus RNAs in a hepatoma cell line. Science 285: 110-113; Krieger et al. (2001), Enhancement of hepatitis C virus RNA replication by cell culture-adaptive mutations. J. Virol. 75: 46144624; and Vrolijk et al. (2003), A replicon-based bioassay for the measurement of interferons in patients with chronic hepatitis C. J. Virol. Meth. 110: 201-209, each herein incorporated by reference with regard to the ET replicon line), includes Huh7 cells stably transfected with HCV genotype 1b strain Con1 replicon. This replicon expresses the firefly luciferase, has the coding region for ubiquitin inserted upstream of the neomycin gene, and carries three cell culture-adaptive mutations that enhance RNA replication cooperatively (E1202G, T1280I, and S2197P). The ET cell line was obtained under licence from: R^{e}BLikon GmbH, Nach dem alten Schloss 22, 55239 Gau-Odemheim, Germany.

Cells are maintained at 37° C., 5% CO₂, in DMEM with 10% FCS, 1× penicillin/streptomycin, 1× non-essential amino acids, 0.5 mg/mL Geneticin. For passage, cells are washed once with PBS, and incubated at 37° C. with sufficient trypsin (0.05%)/versene to cover the monolayer. Once detached from the flask, cells are mixed with several mLs of DMEM+10% FBS to inactivate the trypsin, then diluted appropriately (usually 1 in 5 or 1 in 10) with sufficient medium to pass to a new flask. For a T225 flask, 40-50 mLs total volume of cells+medium is typically used. To prevent the density dependent drop in replicon RNA, the ET cell line is maintained at sub-confluent (60-80%) levels.

Compounds are diluted in DMSO across the first 10 wells of a Costar V-bottom 96-well plate using a Biomek 2000 Workstation (Beckman-Coulter), with 0.5 log₁₀ dilution intervals. DMSO only is added to the last two columns of the plate. The top concentration of drug in this master plate is typically 2.5 mM. A 1/5 dilution of the compounds is made by adding DMEM+10% FBS with a multichannel pipettor, giving a top concentration of 500 μM. 5 μL of diluted compounds is transferred into black Costar flat bottom 96-well daughter plates using a RapidPlate workstation (Zymark).

Cells are grown to approximately 80% confluency and trypsinised as described above. Cells are counted in a Levy Hemocytometer and diluted to 20,000 cells/ml in DMEM containing 10% FBS, 1× penicillin/streptomycin, 1×NEAAs. (Note: Geneticin is omitted for the assay).

245 μL cell suspension is added to the first 11 columns of the daughter plates prepared as above using a Multidrop dispenser (Titertek). Thus, 5,000 cells are added per well, and the final top concentration of each compound is 10 μM. The twelfth column of each plate is used to generate background readings for the assay. Plates are incubated at 37° C., 5% CO₂ for 72 hours.

Luciferase Assay

Steady-Glo reagent is prepared according to the manufacturer's instructions, by mixing the Steady-Glo Buffer and Steady-Glo substrate Buffer at room temperature. Medium is removed from the 96-well plates containing ET cells treated with compound using a multi-channel aspirator. 100 μl of Steady-Glo reagent is added to each well using a multichannel pipettor or Multidrop dispensor. Plates are incubated at room temperature for 5 minutes to ensure complete lysis, and mixed by gentle shaking.

Luciferase activity is read using a Topcount (PE Biosystems), with a 1-second read-time per well. Data are analyzed and IC₆₀ curves generated using RoboFit software.

The compounds herein described are believed to exhibit useful HCV activity as determined by the herein described assay. Although specific IC₅₀ values are given for certain of the present compounds, these values should be considered exemplary. Those skilled in the art will appreciate the variability in performing and recording data using the biological activity assays that are herein described. Table 1, below, provides specific values for exemplary purposes:

TABLE 1

| | HCV activity (ET replicon assay) | |
|---|---|---|
| Example No | IC₅₀ | |
| 14A | 17 | nM |
| 29 | 6 | nM |
| 36B | <30 | nM |
| 38 | 7 | nM |
| 45B | 6 | nM |
| 46B | 16 | nM |

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method for the treatment or prophylaxis of Flaviviridae viruses through administration of a compound of formula (I): 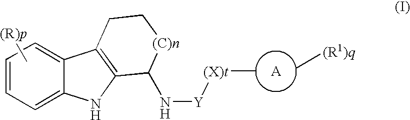
wherein:
n is 0, 1, or 2;
t is 0 or 1;
X is —NH—, —O—, —R¹⁰—, —OR¹⁰—, —R¹⁰O—, —R¹⁰OR¹⁰—, —NR¹⁰—, —R¹⁰N—, —R¹⁰NR¹⁰—, —R¹⁰S(O)ₘ—, or —R¹⁰S(O)ₘR¹⁰—;
Y is —C(O)— or —S(O)ₘ—;
each R is the same or different and is independently selected from the group consisting of
halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰)NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each R¹ is the same or different and is independently selected from the group consisting of halogen, haloalkyl, alkyl, alkenyl, alkynyl, cycloalkyl, cycloalkenyl, —R¹ cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
p and q are each independently selected from 0, 1, 2, 3, 4, or 5;
each of R² and R³ are the same or different and are independently selected from the group consisting of H, alkyl, alkenyl, alkynyl, cycloalkyl, cycloalkenyl, —R¹⁰cycloalkyl, —R¹⁰H, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group;
Het represents a 5- or 6-membered heterocyclyl or heteroaryl group;
ring A is aryl or heteroaryl;
provided that when the A ring is aryl, t is 0, and Y is SO₂, then p is not 0; and
salts and solvates thereof.

2. The method of claim 1 wherein the virus is a flaviviruses, a pestiviruses, or a hepaciviruses.

3. The method of claim 2 wherein the virus is associated with a human disease selected from dengue fever, yellow fever, west nile virus, and HCV.

4. The method of claim 3 wherein method is for the treatment or prophylaxis of HCV infection.

5. The method of claim 1 wherein alkyl is C₁-C₆ alkyl, alkoxy is C₁-C₆ alkoxy, haloalkyl is C₁-C₆ haloalkyl, alkylene is C₁-C₆ alkylene, and alkenylene is C₁-C₆ alkenylene.

6. The method of claim 1 wherein t is 0 and Y is —C(O)—.

7. The method of claim 1 wherein t is 0 and Y is —S(O)ₘ—.

8. The method of claim 1 wherein t is 1, Y is —C(O)—, and X is —NH—, —O—, —R¹⁰—, or —OR¹⁰—.

9. The method of claim 1 wherein t is 1, Y is —S(O)ₘ—, and X is —NH—, —O—, —R¹⁰—, or —OR¹⁰—.

10. The method of claim 1 wherein n is 1.

11. The method of claim 1 wherein p is 1 or more and R is selected from halogen, alkyl, haloalkyl, —OR², —NR²R³, —C(O)R², —CO₂R², cyano, nitro, or azido.

12. The method of claim 11 wherein R is halogen, alkyl, haloalkyl.

13. The method of claim 12 wherein R is substituted para to the depicted N atom.

14. The method of claim 13 wherein R is halogen.

15. The method of claim 14 wherein R is Br or Cl.

16. The method of claim 1 wherein q is 1 or more and R¹ is selected from halogen, alkyl, haloalkyl, —OR²—NR²R³, —C(O)R², —CO₂R², Ay, Het, cyano, nitro, or azido.

17. The method of claim 16 wherein R¹ is selected from halogen, alkyl, haloalkyl, —OR², —NR²R³, —C(O)R², —CO₂R², or cyano.

18. The method of claim 17 wherein R² and R³ each are C₁-C₆ alkyl.

19. The compound of claim 16 wherein R¹ is selected from halogen, alkyl, or —OR².

20. The method of claim 19 wherein said halogen is fluoro or chloro, said alkyl is methyl, and said —OR² is alkoxy.

21. The method of claim 1 wherein the A ring is aryl.

22. The method of claim 21 wherein the A ring is phenyl.

23. The method of claim 22 wherein q is 1 or more and R¹ is selected from halogen, alkyl, haloalkyl, —OR², —NR²R³, —C(O)R², —CO₂R², Ay, Het, cyano, nitro, or azido.

24. The method of claim 23 wherein q is 1 or more and R¹ is selected from halogen, alkyl, haloalkyl, —OR², —NR²³R²², —C(O)R², —CO₂R², or cyano.

25. The method of claim 1 wherein the A ring is heteroaryl.

26. The method of claim 25 wherein the heteroaryl is pyridyl.

27. The method of claim 26 wherein q is 0 or 1.

28. The method of claim 27 wherein when q is 1, then R¹ is selected from halogen, alkyl, haloalkyl, —OR², —NR²R³, —C(O)R², —CO₂R², Ay, Het, cyano, nitro, or azido.

29. The method of claim 28 wherein when q is 1, then R¹ is selected from halogen, alkyl, haloalkyl, —OR², —NR²R³, —C(O)R², —CO₂R² or cyano.

30. The method of claim 1 wherein p is 1, R is halogen, n is 1, Y is —C(O)—, t is 0, ring A is heteroaryl, and q is 0.

31. The method of claim 30 wherein R is chloro and ring A is pyridyl.

32. The method of claim 1 wherein the compound is selected from:

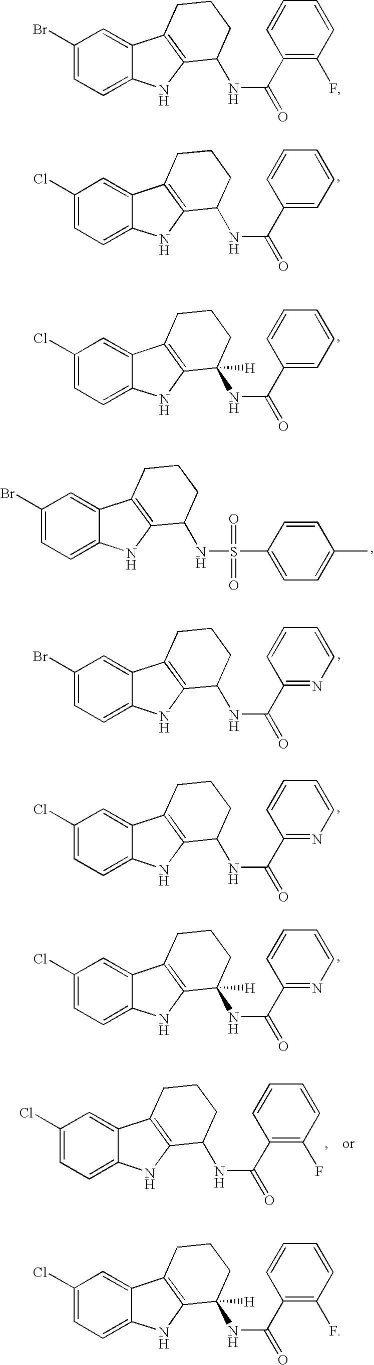

33. The use of a compound of formula (I): 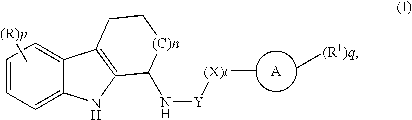
wherein:
n is 0, 1, or 2;
t is 0 or 1;
X is —NH—, —O—, —R¹⁰—, —OR¹⁰—, —R¹⁰O—, —R¹⁰OR¹⁰—, —NR¹⁰—, —R¹⁰N—, —R¹⁰NR¹⁰—, —R¹⁰S(O)ₘ—, or —R¹⁰S(O)ₘR¹⁰;
Y is —C(O)— or —S(O)ₘ—;
each R is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each R¹ is the same or different and is independently selected from the group consisting of halogen, haloalkyl, alkyl, alkenyl, alkynyl, cycloalkyl, cycloalkenyl, —R¹⁰cycloalkyl, Ay, —NHR¹⁰Ay, Het, —NHHet, —NHR¹⁰Het, —OR², —OAy, —OHet, —R¹⁰OR², —NR²R³, —NR²Ay, —R¹⁰NR²R³, —R¹⁰NR²Ay, —R¹⁰C(O)R², —C(O)R², —CO₂R², —R¹⁰CO₂R², —C(O)NR²R³, —C(O)Ay, —C(O)NR²Ay, —C(O)Het, —C(O)NHR¹⁰Het, —R¹⁰C(O)NR²R³, —C(S)NR²R³, —R¹⁰C(S)NR²R³, —R¹⁰NHC(NH)NR²R³, —C(NH)NR²R³, —R¹⁰C(NH)NR²R³, —S(O)₂NR²R³, —S(O)₂NR²Ay, —R¹⁰SO₂NHCOR², —R¹⁰SO₂NR²R³, —R¹⁰SO₂R², —S(O)ₘR², —S(O)ₘAy, cyano, nitro, or azido;
each m independently is 0, 1, or 2;
each R¹⁰ is the same or different and is independently selected from alkylene, cycloalkylene, alkenylene, cycloalkenylene, and alkynylene;
p and q are each independently selected from 0, 1, 2, 3, 4, or 5;
each of R² and R are the same or different and are independently selected from the group consisting of H, alkyl, alkenyl, alkynyl, cycloalkyl, cycloalkenyl, —R¹⁰cycloalkyl, —R¹⁰OH, —R¹⁰(OR¹⁰)_{w}, and —R¹⁰NR⁴R⁵;
w is 1-10;
each of R⁴ and R⁵ are the same or different and are independently selected from the group consisting of alkyl, cycloalkyl, alkenyl, cycloalkenyl, and alkynyl;
Ay represents an aryl group;
Het represents a 5- or 6-membered heterocyclyl or heteroaryl group;
ring A is aryl or heteroaryl;
provided that when the A ring is aryl, t is 0, and Y is SO₂, then p is not 0; and
salts and solvates thereof,
in the manufacture of a medicament for use in the treatment or prophylaxis of viruses belonging to Flaviviridae.

34. The use of claim 33 wherein the virus is a flavivirus, a pestivirus, or a hepacivirus.

35. The use of claim 34 wherein the virus is associated with a human disease or condition is selected from dengue fever, yellow fever, West Nile virus, and HCV.

36. The use of claim 35 wherein the disease or condition is HCV.

37. A compound selected from:
N-(6-Chloro-2,3,4,9-tetrahydro-1H-carbazol-1-yl)-2-pyrazinecarboxamide;
N-(6-Chloro-2,3,4,9-tetrahydro-1H-carbazol-1-yl)-6-methyl-2-pyridinecarboxamide;
N-(6-Chloro-2,3,4,9-tetrahydro-1H-carbazol-1-yl)-5-isoxazolecarboxamide;
N-(6-Chloro-2,3,4,9-tetrahydro-1H-carbazol-1-yl)-2-furancarboxamide;
N-(6-Chloro-2,3,4,9-tetrahydro-1H-carbazol-1-yl)-3,5-dimethyl-4-isoxazolecarboxamide;
N-(6-Chloro-2,3,4,9-tetrahydro-1H-carbazol-1-yl)-6-fluoro-2-pyridinecarboxamide;
N-(6-Chloro-2,3,4,9-tetrahydro-1H-carbazol-1-yl)-3-methylpyridine-2-carboxamide;
N-[2-({2-[(2-Aminoethyl)oxy]ethyl}oxy)ethyl]-N′-(6-chloro-2,3,4,9-tetrahydro-1H-carbazol-1-yl)-1,4-benzenedicarboxamide;
Methyl 6-{[(6-chloro-2,3,4,9-tetrahydro-1H-carbazol-1-yl)amino]carbonyl}-3-pyridinecarboxylate;
6-{[(6-Chloro-2,3,4,9-tetrahydro-1H-carbazol-1-yl)amino]carbonyl}-3-pyridinecarboxylic acid;
1,1-Dimethylethyl [2-({2-[(2-{[(6-{[(6-chloro-2,3,4,9-tetrahydro-1H-carbazol-1-yl)amino]carbonyl}-3-pyridinyl)carbonyl]amino}ethyl)oxy]ethyl}oxy)ethyl]carbamate;
N⁵-[2-({2-[(2-Aminoethyl)oxy]ethyl}oxy)ethyl]-N²-(6-chloro-2,3,4,9-tetrahydro-1H-carbazol-1-yl)-2,5-pyridinedicarboxamide; and
N²-(6-Chloro-2,3,4,9-tetrahydro-1H-carbazol-1-yl)-N⁵-(2-{[2-({2-[(phenylcarbonyl)amino]ethyl}oxy)ethyl]oxy}ethyl)-2,5-pyridinedicarboxamide,
including salts and solvates thereof.

38. (canceled)

39. A pharmaceutical composition comprising a compound according to claim 37, and a pharmaceutically acceptable carrier.

40. A compound according to claim 37 for use as an active therapeutic substance.

41. A compound according to claim 37 for use in the treatment or prophylaxis of diseases and conditions caused by viruses belonging to Flaviviridae.

42. The compound according to claim 41 wherein the virus is a flavivirus, a pestivirus, or a hepacivirus.

43. The compound of claim 42 wherein the virus is associated with a disease or condition is selected from dengue fever, yellow fever, west nile virus, and HCV.

44. The compound of claim 43 wherein the condition or disease is HCV.

45. Use of a compound according to claim 37 in the manufacture of a medicament for use in the treatment or prophylaxis of viruses belonging to Flaviviridae.

46. The use according to claim 45 wherein the virus is a flavivirus, a pestivirus, or a hepacivirus.

47. The use according to claim 46 wherein the virus is associated with a human disease or condition selected from is dengue fever, yellow fever, west nile virus, or and HCV.

48. The use according to claim 47 wherein the disease or condition is HCV.

49. A method for the treatment or prophylaxis of viruses belonging to Flaviviridae comprising administering one or more of a compound of claim 37.

50. The method of claim 49 wherein the virus is a flaviviruses, a pestiviruses, or a hepaciviruses.

51. The method of claim 50 wherein the virus is associated with a human disease selected from dengue fever, yellow fever, west nile virus, and HCV.

52. The method of claim 51 wherein method is for the treatment or prophylaxis of HCV infection.
